(19) United States
(12) Patent Application Publication
(10) Pub. No.: US 2014/0127235 A1
(43) Pub. Date: May 8, 2014

(54) TARGETING HK alpha FOR CONTROLLING BLOOD PRESSURE AND REGULATING ELECTROLYTE BALANCE

(71) Applicant: UNIVERSITY OF FLORIDA RESEARCH FOUNDATION, INC., Gainesville, FL (US)

(72) Inventor: Charles Wingo, Gainesville, FL (US)

(21) Appl. No.: 14/110,197

(22) PCT Filed: Apr. 6, 2012
(86) PCT No.: PCT/US2012/032555

§ 371 Date: Dec. 16, 2013

Related U.S. Application Data
(60) Provisional application No. 61/472,604, filed on Apr. 6, 2011.
(60) Provisional application No. 61/472,862, filed on Apr. 7, 2011.
(60) Provisional application No. 61/493,446, filed on Jun. 4, 2011.

(51) Int. Cl. C12Q 1/68; G01N 33/573

(52) U.S. Cl. CPC C12Q 1/6883; G01N 33/573
USPC 424/172.1; 435/7.21; 435/18; 435/6.12; 536/23.1; 536/24.5; 530/300; 530/389.1; 514/44 R; 514/44 A; 514/15.7

(57) ABSTRACT

Disclosed herein are novel methods for controlling blood pressure that involve targeting HK alpha. Also, disclosed are novel compositions for treating elevated blood pressure that include administering an agent that disrupts HK alpha expression or activity. Methods of screening novel antihypertensives are disclosed as well.

### BACKGROUND

Mineralocorticoid excess represents the most common endocrine form of hypertension and is poorly responsive to typical antihypertensive medications. With improved diagnostic criteria, the prevalence of mineralocorticoid-dependent hypertension is estimated to be as much as ˜5-20% of hypertensive patients.¹⁻³ A major contributing factor to mineralocorticoid-induced hypertension is increased Na⁺ reabsorption by the kidney. Specifically, mineralocorticoids increase expression and activity of the apical epithelial Na⁺ channel and the basolateral Na⁺,K⁺-ATPase in principal cells of the renal collecting duct to drive net Na⁺ reabsorption.⁴ Mineralocorticoids also stimulate H⁺ secretion by the collecting duct, in part by stimulating the activity of apical H⁺-ATPases,^{5, 6} but the effect of mineralocorticoids on H⁺,K⁺-ATPase proton transport activity and expression in these segments has not been determined.

The renal H⁺,K⁺-ATPases are known to localize to the apical membrane of intercalated cells in the collecting duct.⁷ H⁺,K⁺-ATPases are composed of a catalytic α subunit and regulatory β subunit and two different α subunits, HKα₁ and HKα₂, are expressed in the kidney. Only a few studies have investigated the effect of mineralocorticoids on renal H⁺,K⁺-ATPases, and most of these studies focused on acute mineralocorticoid effects (1-2 days) and have not directly measured proton secretion.⁸⁻¹¹

### BRIEF DESCRIPTION OF THE DRAWINGS

FIG. 1. DOCP increased H⁺,K⁺-ATPase-mediated H⁺ secretion (J_{H}) in intercalated cells (ICs) of the mouse collecting duct. J_{H} (H⁺/min) in response to an acute intracellular acid load was measured in ICs of microperfused inner cortical collecting duct from control mice and those treated with DOCP for eight days using the pH sensitive dye, BCECF-AM. Data are presented as mean±SEM and were analyzed by Student's t-test. * P<0.05 compared to control; n shown as cells (tubules).

FIG. 2. DOCP increased H⁺,K⁺-ATPase-mediated H⁺ secretion (J_{H}) in A-type intercalated cell (ICs) of the mouse collecting duct. J_{H} (H⁺/min) in response to an acute intracellular acid load was measured in ICs of microperfused inner cortical collecting duct from control mice and those treated with DOCP for eight days using the pH sensitive dye, BCECF-AM. A- and B-type ICs were differentiated by response to peritubular Cl⁻ removal/return. Data are presented as mean±SEM. The effect of DOCP in each cell type was analyzed using a Student's t-test. * P<0.05 compared to control in the same cell type; n shown as cells (tubules).

FIG. 3. High K⁺ diet abrogated the increase in medullary HKα₂ expression with DOCP treatment in mice. Real time PCR was performed to quantify relative mRNA expression for (A) HKα₁ and (B) HKα₂ in cortex, outer medulla, and inner medulla of control mice and those treated with DOCP for eight days on a normal diet. Relative mRNA expression was also determined for (C) HKα₁ and (D) HKα₂ in cortex, outer medulla, and inner medulla of control and DOCP-treated mice on a high K⁺ (5%) diet. Expression was set relative to β-actin. Fold changes (2̂^{-ΔΔCt}) in expression were calculated and set to %, with control set at 100%. Data are presented as mean±SEM and expression with DOCP treatment was compared to control levels by Student's t-test. * P<0.05 compared to control; n=7-10 for normal diet and n=4 for high K⁺ diet.

FIG. 4. Comparisons of body weight and blood chemistry of wild type, HKα₁^{-/-} and HKα_{1,2}^{-/-} mice treated with DOCP are shown. All data shown are from the eighth day of DOCP treatment. (A) (A) Body weight change (%) is shown as the percent change in body weight from day zero. Arterial blood samples were collected from the aorta and (B) blood [Na⁺], (C) [K⁺], (D) [Cl⁻], and [HCO₃⁻] were measured on a clinical blood gas analyzer. N=10-11 WT and N=5 HKα₁^{-/-} and HKα_{1,2}^{-/-} mice. Data are presented as mean±s.e.m. and were analyzed by one-way ANOVA followed by post-hoc Student-Newman-Keuls test. * P<0.05 compared to WT; ψP<0.05 compared to HKα₁^{-/-} mice.

FIG. 5. DOCP treatment differentially altered urinary Na⁺ and K⁺ retention in wild type (WT) and HKα null mice. (A) Urinary volume, (B) Na⁺, (C) and K⁺ retention were measured in WT, HKα₁^{-/-} and HKα_{1,2}^{-/-} mice from the day preceding treatment (control) and over an eight day period of DOCP treatment. Urinary electrolyte retention (μEq) was calculated as the urinary excretion per day subtracted from dietary intake on that day. (D-F) Comparisons of control urine values versus only day eight of DOCP treatment are shown. All data were analyzed by a two-way repeat measure ANOVA with post hoc Student-Newman-Keuls test and are shown as mean±s.e.m. P<0.05 considered significant. For comparison of genotype within day, * denotes significant difference from WT and ψ denotes significant difference from HKα₁^{-/-} mice. For comparison of genotype over the time course, ** denotes significant difference from WT and ψ denotes significant difference from HKα₁^{-/-} mice. ω denotes significant difference from control. WT, N=10; HKα₁^{-/-}, N=5; HKα_{1,2}^{-/-}, N=5.

FIG. 6. Fecal electrolyte excretion in control and DOCP-treated wild type (WT) and HKα null mice is shown. (A) Fecal output, (B) Na⁺, and (C) K⁺ excretion were measured in WT, HKα₁^{-/-}, and HKα_{1,2}^{-/-} mice on the day preceding treatment (control) and on day eight of DOCP treatment. Data are shown as mean±s.e.m. and were analyzed by two-way repeat measure ANOVA with post hoc Student-Newman-Keuls test; P<0.05 considered significant. * denotes significant difference from WT; ψ denotes significant difference from HKα₁^{-/-} mice; ω denotes significant difference from control in the same genotype. WT, N=11; HKα₁^{-/-}, N=4; HKα_{1,2}^{-/-}, N=5-6.

FIG. 7. HKα null mice display disturbances in overall electrolyte balance with DOCP treatment. Overall (A) Na⁺ and (B) K⁺ balance (μEq) are shown for wild type (WT), HKα₁^{-/-}, and HKα_{1,2}^{-/-} mice on the day preceding treatment (control) and on day eight of DOCP treatment. Overall electrolyte balance was calculated as the amount of the electrolyte excreted in the urine and feces subtracted from dietary intake of that electrolyte. Data are shown as mean±s.e.m. and were analyzed by two-way repeat measure ANOVA with post hoc Student-Newman-Keuls test or one-way repeat measure ANOVA with post-hoc Tukey test; P<0.05 considered significant. * denotes significant difference from WT; ψ denotes significant difference from HKα₁^{-/-} mice; ω denotes significant difference from control in the same genotype. WT, N=10; HKα₁^{-/-}, N=4; HKα_{1,2}^{-/-}, N=5.

FIG. 8. ENaC subunit mRNA expression is similar in WT and HKα_{1,2}^{-/-} mice. Real time PCR was used to assess α-, β-, and γ-ENaC mRNA expression in kidney cortex (Ctx) and medulla (Med) from WT and HKα_{1,2}^{-/-} mice fed a normal diet. Expression was set relative to β-actin. Fold changes (2^{-ΔΔCt}) in expression were calculated, with WT set at 1. Data are presented as mean±SEM and analyzed by Student's t-test. N=6-8.

FIG. 9. Medullary αENaC protein expression is reduced in HKα_{1,2}^{-/-} mice. Western blot analysis was used to assess α- and γENaC protein expression in total protein fractions from renal medulla of WT and HKα_{1,2}^{-/-} mice fed a normal gel diet ad libitum. A) A representative blot is shown for α- and γENaC protein (˜85 kDa) expression with β-actin (˜42 kDa) used a loading control. B) Densitometry analysis of blots for α- and γENaC protein expression, corrected for β-actin levels, with WT expression set to 100%. Data are shown as mean±SEM and were analyzed by Student's t-test. † denotes P<0.05 versus WT. N=5 for αENaC and N=3 for γENaC.

FIG. 10. HKα_{1,2}^{-/-} mice display altered appetite and urine volume on a normal diet. A) Food consumption, B) water intake, C) urine volume, and D) urine osmolality were measured in WT and HKα_{1,2}^{-/-} mice fed a normal gel diet ad libitum for one week. Data are an average of 3 days (day 5 to 7) shown as mean±SEM and were analyzed by Student's t-test. * denotes P<0.05 versus WT. N=3.

FIG. 11. HKα_{1,2}^{-/-} mice lost considerable body weight when food restricted. Body weight, shown as percent change from day 0, was measured in WT and HKα_{1,2}^{-/-} mice pair fed (food restricted) or fed a normal gel diet ad libitum for one week. Data are shown as mean±SEM and differences between genotypes were analyzed by Student's t-test. * denotes P<0.05 versus WT on the same diet. N=3-4.

FIG. 12. Food restriction (pair feeding) caused HKα_{1,2}^{-/-} mice to exhibit augmented urinary aldosterone excretion. Urine aldosterone, shown as ng excreted per day, was measured in male WT and HKα_{1,2}^{-/-} mice fed ad libitum or pair fed a normal gel diet. Data are shown as mean±SEM and were analyzed by Student's t-test. * denotes P<0.05 versus WT on the same diet. N=3-4.

FIG. 13. Example 48 hour tracings of systolic blood pressure (BP), heart rate (HR), and locomotor activity (LA) in a WT (solid line) and a HKα_{1,2}^{-/-} mouse (dashed line) during normal and DOCP treatment.

FIG. 14. Three of seven DOCP-treated H,Kα_{1,2}^{-,-} mice (dashed line) died by day 47; whereas all nine WT (solid line) mice survived. A second DOCP treatment

FIG. 15. Dietary K⁺ depletion caused excessive fecal (not urinary) K+ excretion in HKα_{1,2}^{-/-} mice. A) Data (μEq) are displayed as 24 hour urinary K+ excretion subtracted from 24 hour dietary K+ intake on day 8 of either a control or dietary K+ depleted diet. B) Fecal K+ excretion (μEq/g stool) is shown for day 4 of the control diet and C) day 4 and 8 of the K+ depleted diet. Data are presented as mean±SEM and were analyzed by either a one-way or two-way ANOVA followed by a post hoc Student-Newman-Keuls or Holm-Sidak test, respectively (N=6-8).

FIG. 16. Dietary K+ depletion caused urinary Na+ retention in WT and HKα_{1,2}^{-/-} mice. A) Body weight change is shown as the difference between day 8 and day 0. B) Data (μEq) are displayed as 24 hour urinary Na+ excretion subtracted from 24 hour dietary Na+ intake on day 8 of either a control or dietary K+ depleted diet. All data are presented as mean±SEM and were analyzed by two-way ANOVA followed by a post hoc Holm-Sidak test (N=6-8).

FIG. 17. HKα_{1,2}^{-/-} mice did not exhibit greater urinary K+ or Na+ loss than WT at an earlier time point during dietary K+ depletion. Data (μEq) are displayed as 24 hour urinary A) K+ or B) Na+ excretion subtracted from 24 hour dietary K+ or Na+ intake on the day before a K+ depleted diet (control diet) and on the following four days during dietary K+ depletion. Data are presented as mean±SEM and were analyzed by two-way repeated measure ANOVA followed by a post hoc Holm-Sidak test (N=4-5).

### DETAILED DESCRIPTION

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

Aldosterone and other mineralocorticoids, such as desoxycorticosterone cause sodium retention and hypertension, metabolic alkalosis and hypokalemia. We have identified one gene that is necessary for the sodium retention, weight gain, and electrolyte abnormalities (metabolic alkalosis and hypokalemia) when mice are ingesting normal diet. These studies use mice that are deficient in the genes that encode for either the HK alpha-1 or the HK alpha-1 and HK alpha-2 subunits and demonstrate that only mice that are deficient in both the genes that encode for HK alpha-1 (ATP4A) and HK alpha-2 (ATP12A) fail to respond to the mineralocorticoid desoxycorticosterone. Mice that are deficient in the gene ATP4A which encodes for the HK alpha-1 subunit of the H,K-ATPase expressed in the stomach and in the kidney exhibit either a normal response compared to wild type animals or an exaggerated (increased) response to the administration of desoxycorticosterone. The studies demonstrate that the gene that encodes for HK alpha-2 (ATP12A) is responsible for the lack of fluid retention, sodium retention, or the metabolic alkalosis and hypokalemia that occurs in normal mice when given the mineralocorticoid desoxycorticosterone.

The treatment of both sodium retention and systemic arterial hypertension are common problems in patients with cardiovascular, renal, or hepatic organ dysfunction (heart failure chronic kidney failure chronic liver disease), or diabetes mellitus. Hypertension is a major contributor to cardiovascular disease, and cardiovascular disease is the leading cause of death in the United States and most countries in Europe and industrialized countries. Excessive, autonomous secretion of aldosterone (primary hyperaldosteronism) occurs in approximately 20% of patients with refractory hypertension and is associated with significant hypokalemia and metabolic alkalosis. Hypokalemia is a serious risk factor for cardiac arrhythmias in patients with coronary artery disease (ischemic heart disease, as manifest by angina pectoris, myocardial infarction, and sudden death).

Sodium retention can occur without hypertension and frequently complicates therapy in patients with left heart failure, frequently referred to as congestive heart failure or CHF. Sodium retention is also common in patients with renal disease, advanced liver disease, and patients with diabetes mellitus. Patients with diabetes mellitus may either require the treatment with insulin (insulin-dependent, or type I diabetics) or treatment with oral hypoglycemic drugs, such as the thiazolidinediones, which are PPARgamma agonists (non-insulin-dependent diabetics). Sodium and fluid retention is a known complication of PPARgamma agonists and experimental animal studies suggest that this fluid retention occurs within the collecting duct, specifically where the HK alpha-2 H,K-ATPases are expressed in the kidney.

The inventors have surmised that HK alpha (typically alpha 2) may be targeted as a therapy for elevated blood pressure and other related cardiovascular diseases. Thus, according to one embodiment, the invention pertains to a method of treating elevated blood pressure that comprises blocking activity or expression of HK alpha-2.

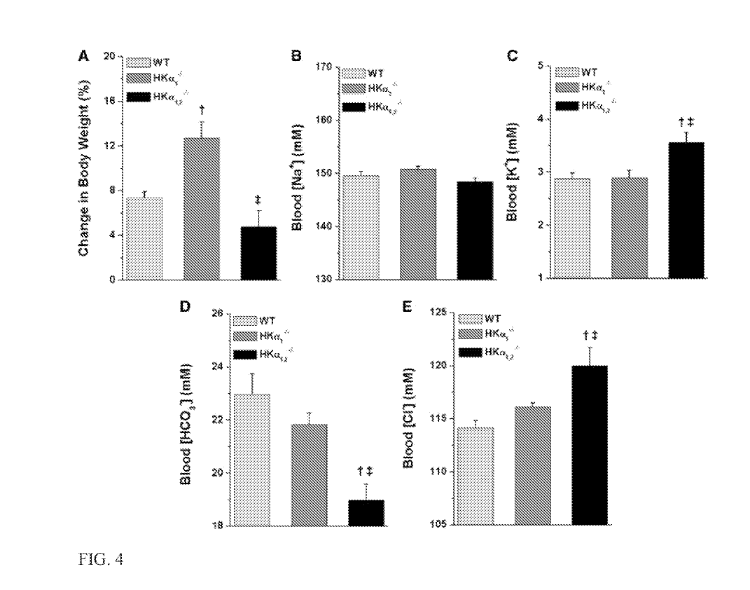

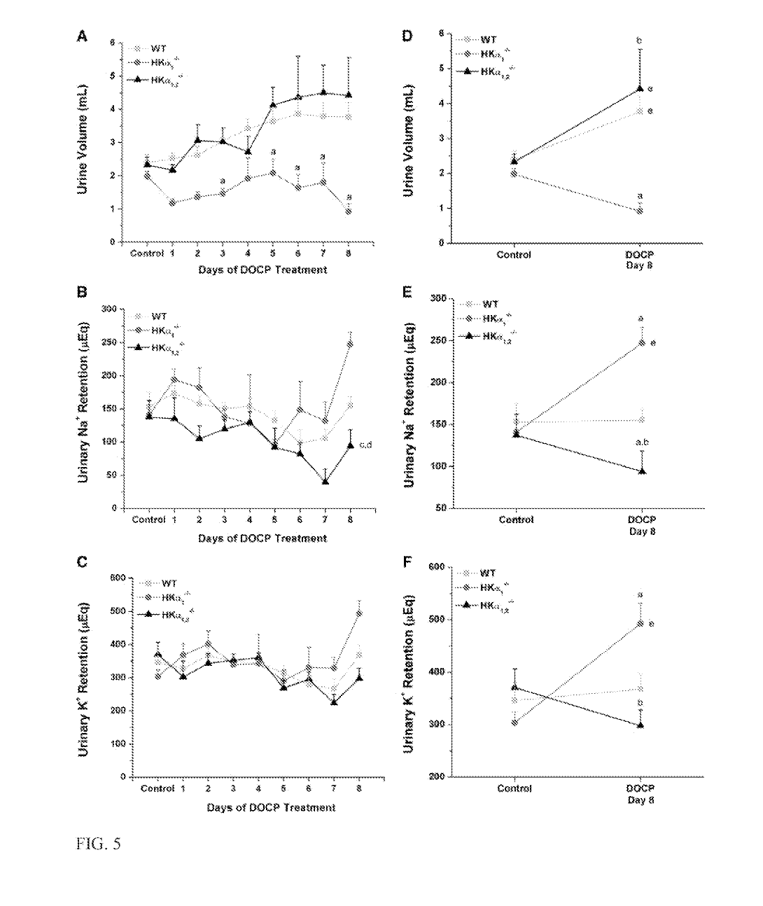

According to another embodiment, the invention pertains to a pharmaceutical composition for the treatment of elevated blood pressure comprising a therapeutic agent which regulates the activity of the holoenzyme which has an absolute requirement for the HK alpha polypeptide for function of the enzyme, wherein said therapeutic agent is i) a small molecule, ii) an RNA molecule, iii) an antisense oligonucleotide, iv) a polypeptide, v) an antibody, or vi) a ribozyme, targeted to disrupt expression of HK alpha or disrupt activity of HK alpha. In a specific embodiment, the RNA molecule is siRNA, microRNA, shRNA engineered to bind to an HK alpha sequence mRNA. J Am Soc Nephrol. 2001 December; 12(12):2554-64 and Kidney International (1999) 56, 1029-1036 are cited for sequence information, and specifically highly conserved regions of HK alpha sequences that are distinct from other members of the family of proteins (Na,K-ATPase, Ca-ATPase, etc). Also, Zhang et al., JASN, 2001, 12:2554-2564 teaches the sequence of the muringe HK α2 gene, (see FIG. 4). The sequences disclosed therein are non-limiting examples of sequences that can be targeted for therapeutic applications as well as for screening assays, and are incorporated herein by reference

According to another embodiment, the invention pertains to a recombinant cell host containing a purified HK alpha polynucleotide or a recombinant vector comprising a HK alpha polynucleotide.

In some embodiments, the term “elevated blood pressure” refers to a systemic arterial systolic blood pressure of 121 mm or higher in a subject.

Many of the embodiments of the subject invention make reference to particular methods of inhibiting or disruption of genetic expression. Based on the teachings herein, methods of expression include but are not limited to siRNA; ribozyme(s); antibody(ies); antisense/oligonucleotide(s); morpholino oligomers; microRNA; or shRNA that target expression of the HK alpha-2 protein. The subject invention is not to be limited to any of the particular related methods described. One such method includes siRNA (small interfering/short interfering/silencing RNA). SiRNA most often is involved in the RNA interference pathway where it interferes with the expression of a specific gene. In addition to its role in the RNA interference pathway, siRNA also act in RNA interference-related pathways, e.g., as an antiviral mechanism or in shaping the chromatin structure of a genome.

Another method by which to inhibit expression and to inhibit the expression of HK-alpha in particular is shRNA. ShRNA (short hairpin or small hairpin RNA) refers to a sequence of RNA that makes a tight hairpin turn and is used to silence gene expression via RNA interference. It uses a vector introduced into cells and a U6 or H1 promoter to ensure that the shRNA is always expressed. The shRNA hairpin structure is cleaved by cellular machinery into siRNA which is then bound to the RNA-induced silencing complex. This complex binds to and cleaves mRNAs which match the siRNA that is bound to it.

HK-alpha can also be blocked by subjecting procured cells to an antibody specific to HK-alpha. An antisense nucleotide may also be used to block or inhibit expression, in particular, the expression of HK alpha. Expression may also be inhibited with the use of a morpholino oligomer or phosphorodiamidate morpholino oligomer (PMO). PMOs are an antisense technology used to block access of other molecules to specific sequences within nucleic acid. PMOs are often used as a research tool for reverse genetics, and function by knocking down gene function. This is achieved by preventing cells from making a targeted protein or by modifying splicing of pre-mRNA. One embodiment of the subject invention pertains to a method of treating renal lesions or other places where HKalpha-2 is expressed in a subject in need thereof.

The term “subject” as used herein refers to a human or a non-human mammal. Non-human mammals include, but are not limited to, rodents such as rats and mice, cats, dogs, horses, cattle, goats, sheep or pigs.

As provided by the methods of the invention herein, the term “administering”, “administer” or “administration” with respect to delivery of cells to a subject refers to injecting one or a plurality of cells with a syringe, inserting the stem cells with a catheter or surgically implanting the stem cells. In certain embodiments, the stem cells are administered into a body cavity fluidly connected to a target tissue. In other embodiments, the stem cells are inserted using a syringe or catheter, or surgically implanted directly at the target tissue site. In other embodiments, the stem cells are administered systemically (e.g., parenterally). In other specific examples, stem cells are administered by intraocular delivery, intramuscular delivery, subcutaneous delivery or intraperitioneal delivery.

As provided by the methods of the invention herein, the term “administering”, “administer” or “administration” with respect to delivery of a HK-alpha blocking agent to a subject refers to parenteral administration, intraperitoneal, intramuscular, intraocular administration including transcleral administration, and intravitreal injection; transdermal administration, oral administration, intranasal administration, direct delivery to a target site or delivery to a body cavity in fluid communication with a target site.

As used herein, the terms “antisense oligonucleotide” and “antisense oligomer” are used interchangeably and refer to a sequence of nucleotide bases and a subunit-to-subunit backbone that allows the antisense oligonucleotide or oligomer to hybridize to a target sequence in an RNA by Watson-Crick base pairing, to form an RNA:oligomer heteroduplex within the target sequence. The oligomer may have exact sequence complementarity to the target sequence or near complementarity. Such antisense oligomers may block or inhibit translation of the mRNA containing the target sequence, or inhibit gene transcription, may bind to double-stranded or single stranded sequences, and may be said to be “directed to” a sequence with which it hybridizes.

The term “coadministering” or “concurrent administration”, when used, for example with respect to two or more HK alpha blocking agents or a blocking agent and a known antihypertensive agent, refers to administration of the agents such that both can simultaneously achieve a physiological effect. The agents, however, need not be administered together. In certain embodiments, administration of one can precede administration of the other, however, such coadministering typically results in the agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

According to other embodiments, provided is a method wherein differences (mutations) in the composition of HKalpha 2 gene or the HKbeta gene or the NaKbeta1 gene are determined that give rise to altered protein products of said genes or that detect altered level of expression or activity of said gene.

According to a further embodiment, provided is a method wherein differences (mutations) in the composition of HKalpha 2 gene or the HKbeta gene or the NaKbeta1 gene are determined that give rise to altered protein products of genes for the alpha, beta or gamma ENaC subunits or that detect altered level of expression or activity of these genes.

The following discussion will further describe the screening methods, compositions, and methods for targeting HKalpha enzymes for therapeutic purposes.

### 1. Screening Methods

The present invention provides for screening test compounds which bind to or modulate the activity of a HKalpha polypeptide or bind to and inhibit or affect expression of a HKalpha polynucleotide. A test compound preferably binds to a HKalpha polypeptide. More preferably, a test compound reduces or increases HKalpha activity by at least about 10, preferably about 50, more preferably about 75, 90, or 100% relative to the absence of the test compound. In one example, HKalpha activity measured pertains to the ability to transport ions such as potassium or Hydrogen.

### 1.1. Test Compounds

Test compounds relate to agents that potentially have therapeutic activity, i.e., bind to or modulate the activity of a HKalpha polypeptide or bind to or affect expression of a HKalpha polynucleotide. Test compounds can be pharmacologic agents already known in the art or can be compounds previously unknown to have any pharmacological activity. The compounds can be naturally occurring or designed in the laboratory. They may be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al, Proc. Natl. Acad. Sci. U.S.A. 90, 6909, 1993; Erb et al. Proc. NatL. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994).

### 1.2. High Throughput Screening

Test compounds can be screened for the ability to bind to and inhibit HKalpha polypeptides or polynucleotides or to affect HKalpha activity or HKalpha gene expression using high throughput screening. Using high throughput screening, many discrete compounds can be tested in parallel so that large numbers of test compounds can be quickly screened. The most widely established techniques utilize 96, 384 and 1536-well microtiter plates. The wells of the microtiter plates typically require assay volumes that range from 5 to 500 μl. In addition to the plates, many instruments, materials, pipettors, robotics, plate washers, and plate readers are commercially available to fit these multi-well formats. Alternatively, “free format assays,” or assays that have no physical barrier between samples, can be used.

### 1.3. Binding Assays

For binding assays, the test compound is preferably, but not necessarily, a small molecule which binds to and occupies, for example, the active site of the HKalpha polypeptide, such that normal biological activity is prevented. Examples of such small molecules include, but are not limited to, small peptides or peptide-like molecules as are described below.

In binding assays, either the test compound or the HKalpha polypeptide can comprise a detectable label, such as a fluorescent, radioisotopic, chemiluminescent, or enzymatic label, such as horseradish peroxidase, alkaline phosphatase, or luciferase. Detection of a test compound which is bound to the HKalpha polypeptide can then be accomplished, for example, by direct counting of radioemmission, by scintillation counting, or by determining conversion of an appropriate substrate to a detectable product.

Those skilled in the art equipped with teachings herein will appreciate that there are multiple conventional methods of detecting binding of a test compound. For example, binding of a test compound to a HKalpha polypeptide can be determined without labeling either of the interactants. A microphysiometer can be used to detect binding of a test compound with a HKalpha polypeptide. A microphysiometer (e.g., CYTOSENSOR™) is an analytical instrument that measures the rate at which a cell acidifies its environment using a light-addressable potentiometric sensor (LAPS). Changes in this acidification rate can be used as an indicator of the interaction between a test compound and a HKalpha polypeptide (McConnell et al., Science 257, 19061912, 1992).

In another alternative example, determining the ability of a test compound to bind to a HKalpha polypeptide can be accomplished using a technology such as real-time Bimolecular Interaction Analysis (BIA) (Sjolander & Urbaniczky, Anal Chem. 63, 23382345, 1991, and Szabo et al., Curr. Opin. Struct. Biol. 5, 699705, 1995). BIA is a technology for studying biospecific interactions in real time, without labeling any of the interactants (e.g., BIAcore™) Changes in the optical phenomenon surface plasmon resonance (SPR) can be used as an indication of real-time reactions between biological molecules.

In yet another aspect of the invention, a HKalpha polypeptide can be used as a “bait protein” in a two-hybrid assay or three-hybrid assay (see, e.g., U.S. Pat. No. 5,283,317; Zervos et al., Cell 72, 223232, 1993; Madura et al., J. Biol. Chem. 268, 1204612054, 1993; Bartel et al., BioTechniques 14, 920924, 1993; Iwabuchi et al., Oncogene 8, 16931696, 1993; and Brent WO94/10300), to identify other proteins which bind to or interact with the HKalpha polypeptide and modulate its activity.

In many screening embodiments, it may be desirable to immobilize either the HKalpha polypeptide (or polynucleotide) or the test compound to facilitate separation of bound from unbound forms of one or both of the interactants, as well as to accommodate automation of the assay. Thus, either the HKalpha polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, glass, or magnetic beads). Any method known in the art can be used to attach the HKalpha polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a HKalpha polypeptide (or polynucleotide) can be accomplished in any vessel suitable for containing the reactants. Examples of such vessels include microtiter plates, test tubes, and microcentrifuge tubes.

In specific embodiments, the HKalpha polypeptide may be a fusion protein comprising a domain that allows the HKalpha polypeptide to be bound to a solid support. For example, glutathione S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed HKalpha polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a HKalpha polypeptide (or polynucleotide) or a test compound can be immobilized utilizing conjugation of biotin and streptavidin. Biotinylated HKalpha polypeptides (or polynucleotides) or test compounds can be prepared from biotinNHS(Nhydroxysuccinimide) using techniques well known in the art (e.g., biotinylation kit, Pierce Chemicals, Rockford, 111.) and immobilized in the wells of streptavidin-coated 96 well plates (Pierce Chemical). Alternatively, antibodies which specifically bind to a HKalpha polypeptide, polynucleotide, or a test compound, but which do not interfere with a desired binding site, such as the active site of the HKalpha polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the HKalpha polypeptide or test compound, enzyme-linked assays which rely on detecting an activity of the HKalpha polypeptide, and SDS gel electrophoresis under non-reducing conditions.

Screening for test compounds which bind to a HKalpha polypeptide or polynucleotide also can be carried out in an intact cell. Any cell which comprises a HKalpha polypeptide or polynucleotide can be used in a cell-based assay system. A HKalpha polynucleotide can be naturally occurring in the cell or can be introduced using techniques such as those described above. Binding of the test compound to a HKalpha polypeptide or polynucleotide is determined as described above.

### 1.4. Enzyme Assays

Test compounds can be tested for the ability to increase or decrease the HKalpha activity of a HKalpha polypeptide. Enzyme assays can be carried out after contacting either a purified HKalpha polypeptide, a cell membrane preparation, or an intact cell with a test compound. A test compound which decreases the activity of a HKalpha polypeptide by at least about 10, preferably about 50, more preferably about 75, 90, or 100% is identified as a potential therapeutic agent for use herein.

### 1.5. Gene Expression

In another embodiment, test compounds which increase or decrease HKalpha gene expression are identified. A HKalpha polynucleotide is contacted with a test compound, and the expression of an RNA or polypeptide product of the HKalpha polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of HKalpha mRNA or polypeptide expression in the cells can be determined by methods well known in the art for detecting mRNA or polypeptide. Either qualitative or quantitative methods can be used. The presence of polypeptide products of a HKalpha polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a HKalpha polypeptide.

Such screening can be carried out either in a cell-free assay system or in an intact cell. Any cell which expresses a HKalpha polynucleotide can be used in a cell-based assay system. The HKalpha polynucleotide can be naturally occurring in the cell or can be introduced using techniques such as those described above. Either a primary culture or an established cell line, such as CHO or human embryonic kidney 293 cells, can be used.

### 2. Pharmaceutical Compositions

Aspects also provide pharmaceutical compositions comprising one or more therapeutic agents that are identified by the screening methods provided herein or as are described herein below. Therapeutic agent(s) can be administered to a patient to achieve a therapeutic effect, i.e. useful in modulating HKalpha activity and in turn, treating and/or preventing hypertension. Pharmaceutical compositions of the invention can comprise, for example, therapeutic agents identified by a screening method embodiment described herein, which are identified by their ability to bind to or affect activity of HKalpha polypeptides, or bind to and/or affect expression HKalpha polynucleotides. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other therapeutic agents or treatments.

In addition to the active ingredients, these pharmaceutical compositions can contain suitable pharmaceutically acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the subject.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

The present invention further pertains to the use of novel agents identified by the screening assays described above. Accordingly, it is within the scope of this invention to use a therapeutic agent identified as described herein in an appropriate animal model. For example, an agent identified as described herein (for example, but not limited to, a modulating agent, an antisense nucleic acid molecule, a specific antibody, ribozyme, interfering molecule, or a HKalpha-binding molecule) can be used in an animal model to determine the efficacy, toxicity, or side effects of treatment with such an agent. Furthermore, the present invention includes uses of novel agents identified by the above-described screening assays for treatments as described herein.

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 1×10⁶ cells, more preferably about 1.0 μg of DNA per 16 nmole of liposome delivered to about 1×10⁶ cells, and even more preferably about 2.0 μg of DNA per 16 nmol of liposome delivered to about 1×10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes conventionally used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about

1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

In one embodiment, the delivery system enhances cell targeting, prolongs circulation time, and/or improves membrane permeation, while being biocompatible and biodegradable. Exemplary delivery systems include lipids, peptides, synthetic and natural polymers, viral and non-viral vectors, liposomes, micelles, emulsions, microemulsions, microtubes, and nanotubes. When the HKalpha inhibitor is an siRNA molecule, the delivery system may comprise a regulatory sequence useful in expression constructs/vectors with siRNA. Exemplary regulatory sequences may include a constitutive promoter, an inducible promoter, a tissue-specific promoter, or a combination thereof.

In a particular embodiment, the delivery system comprises a liposome. Liposomes comprising various lipid compositions useful in delivering a HKalpha inhibitor as described herein are known in the art. See Fraley, R., and Papahadjopoulos, D., Trends Biochem. Sci. 6: 77-80). Exemplary liposome preparations are available, such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

In another embodiment, the delivery of the HKalpha inhibitor, typically an interfering molecule, may be accomplished using any one or more of a number of recombinant DNA and gene therapy technologies, including viral vectors. Viral vector methods and protocols are reviewed in Kay et al., Nature Medicine 7:33-40, 2001. Viral vectors useful in the invention include those derived from Adeno-Associated Virus (AAV). An exemplary AAV vector comprises a pair of AAV inverted terminal repeats, which flank at least one cassette containing a promoter which directs expression operably linked to a nucleic acid encoding a molecule that modulates HKalpha. Methods for use of recombinant AAV vectors are discussed, for example, in Tal, J., J. Biomed. Sci. 7:279-291, 2000 and Monahan and Samulski, Gene Therapy 7:24-30, 2000.

In a particular embodiment, the HKalpha inhibitor may be delivered via using a lentivirus. Lentiviruses are a subclass of retroviruses. They have recently been adapted as gene delivery vehicles (vectors) due to their ability to integrate into the genome of non-dividing cells, which is the unique feature of lentiviruses as other retroviruses may only infect dividing cells. The lentivirus may be particularly suitable for in vivo evaluation of an inhibitor. See e.g., Song Y, Zhang Z, Yu X, Yan M, Zhang X, Gu S, et al. (2006). Application of lentivirus-mediated RNAi in studying gene function in mammalian tooth development. Dev Dyn 235:1334-1344.

In yet another embodiment, the delivery system comprises a nanoparticle delivery system such as that disclosed in US Published Patent Application No. 20110091510 (University of Florida Research Foundation), the entirety of which is incorporated by reference.

### 2.1 Determination of a Therapeutically Effective Dose

The determination of a therapeutically effective dose of therapeutic agents identified by a screening method herein is well within the capability of those skilled in the art. A therapeutically effective dose refers to that amount of active ingredient which modulates HKalpha activity compared to that which occurs in the absence of the therapeutically effective dose.

Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Preferably, a therapeutic agent reduces expression of a HKalpha gene or the activity of a HKalpha polypeptide by at least about 10, preferably about 50, more preferably about 75, 90, or 100% relative to the absence of the reagent. The effectiveness of the mechanism chosen to decrease the level of expression of a HKalpha gene or the activity of a HKalpha polypeptide can be assessed such as by hybridization of nucleotide probes to HKalpha-specific mRNA, quantitative RT-PCR, immunologic detection of a HKalpha polypeptide, or measurement of HKalpha activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles.

The combination of therapeutic agents can act synergistically to effect the treatment or prevention of hypertension. Using this approach, one may be able to achieve therapeutic efficacy with lower dosages of each agent, thus reducing the potential for adverse side effects. Any of the therapeutic methods described above can be applied to any subject in need of such therapy.

### 3. Polypeptides

A HKalpha polypeptide of the invention therefore can be a portion of a HKalpha protein, a full-length HKalpha protein, or a fusion protein comprising all or a portion of HKalpha protein. In one embodiment, HKalpha polypeptides according to the invention comprise at least 12, 15, 25, 50, 75, 100, 125, 150, 175, 200, 225, 250, 300, 350, 400, 450, 500, 525, 550, 575, 600, or 625 contiguous amino acids selected from the amino acid sequence shown in SEQ ID NOS: 1, 2, 5 or 7, or other HKalpha sequences disclosed above or known in the art, or a biologically active variant thereof, as defined below. Appendix A sets forth the sequences of SEQ ID NO.s 1-8. SEQ ID NOs 7 and 8 pertain to the polypeptide sequence of HK alpha 1, which could also be sequences used in accordance with the teachings herein.

### 3.1 Biologically Active Variants

HKalpha polypeptide variants which are biologically active also are considered HKalpha polypeptides for purposes of this application. In one embodiment, naturally or non-naturally occurring HKalpha polypeptide variants have amino acid sequences which are at least about 55, 60, 65, or 70, preferably about 75, 80, 85, 90, 96, 96, or 98% identical to the amino acid sequence shown in SEQ ID NOS: 1, 2, 5 or 7, or other HKalpha sequences disclosed above or known in the art, or a fragment thereof. Percent identity between a putative HKalpha polypeptide variant and an amino acid sequence for HKalpha may be determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a HKalpha polypeptide can be found using computer programs well known in the art, such as DNASTAR software. Whether an amino acid change results in a biologically active HKalpha polypeptide can readily be determined by assaying for HKalpha activity as would be readily determined by one skilled in the art.

### 3.2 Fusion Proteins

In some embodiments of the invention, it is useful to create fusion proteins. By way of example, fusion proteins are useful for generating antibodies against HKalpha polypeptide amino acid sequences and for use in various assay systems. For example, fusion proteins can be used to identify proteins which interact with portions of a HKalpha polypeptide. Protein affinity chromatography or library-based assays for protein-protein interactions, such as the yeast two-hybrid or phage display systems, can be used for this purpose. Such methods are well known in the art and also can be used as drug screens.

A HKalpha polypeptide fusion protein comprises two polypeptide segments fused together by means of a peptide bond. For example, the first polypeptide segment can comprise at least 12, 15, 25, 50, 75, 100, 125, 150, 175, 200, 225, or 250 contiguous amino acids of SEQ ID NOS: 1, 2, 5 or 7, or other HKalpha sequence referenced above or known in the art. The first polypeptide segment also can comprise full-length HKalpha protein. The second polypeptide segment can be a full-length protein or a protein fragment. Proteins commonly used in fusion protein construction include galactosidase, glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions. A fusion protein also can be engineered to contain a cleavage site located between the HKalpha polypeptide-encoding sequence and the heterologous protein sequence, so that the HKalpha polypeptide can be cleaved and purified away from the heterologous moiety.

Numerous different kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### 4. Polynucleotides

A HKalpha polynucleotide can be single- or double-stranded and comprises a coding sequence or the complement of a coding sequence for a HKalpha polypeptide. A coding sequence for the HKalpha polypeptides, such as those disclosed by SEQ ID NOS: 1, 2, 5 or 7 are readily ascertainable by one skilled in the art. Examples of polynucleotides include those of SEQ ID NOs. 3, 4, 6 or 8. Degenerate nucleotide sequences encoding HKalpha polypeptides, as well as homologous nucleotide sequences which are at least about 50, 55, 60, 65, 60, preferably about 75, 90, 96, or 98% identical to a particular HKalpha nucleotide sequence are also HKalpha-like enzyme polynucleotides. Percent sequence identity between the sequences of two polynucleotides is determined using computer programs such as ALIGN which employ the FASTA algorithm, using an affme gap search with a gap open penalty of −12 and a gap extension penalty of −2. Complementary DNA (cDNA) molecules, species homologs, and variants of HKalpha polynucleotides which encode biologically active HKalpha polypeptides also are HKalpha polynucleotides.

### 4.1 Identification of Polynucleotide Variants and Homologs

Variants and homologs of the HKalpha polynucleotides described above also are HKalpha polynucleotides. Typically, homologous HKalpha polynucleotide sequences can be identified by hybridization of candidate polynucleotides to known HKalpha polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of the HKalpha polynucleotides disclosed herein also can be identified by making suitable probes or primers and screening cDNA expression libraries. It is well known that the Tm of a double-stranded DNA decreases by 1-1.5° C. with every 1% decrease in homology (Bonner et al, J. Mol. Biol. 81, 123 (1973). Variants of HKalpha polynucleotides or polynucleotides of other species can therefore be identified by hybridizing a putative homologous HKalpha polynucleotide with a polynucleotide having a HKalpha nucleotide sequence or complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to HKalpha polynucleotides or their complements following stringent hybridization and/or wash conditions also are HKalpha polynucleotides. Stringent wash conditions are well known and understood in the art and are disclosed, for example, in Sambrook et al., MOLECULAR CLONING: A LABORATORY MANUAL, 2 nd ed., 1989, at pages 9.50-9.51.

Typically, for stringent hybridization conditions a combination of temperature and salt concentration should be chosen that is approximately 12-20° C. below the calculated Tm (melting temperature) of the hybrid under study. The T, of a hybrid between a HKalpha polynucleotide or a complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na+])+0.41(% G+C)−0.63(% formamide)−600/l), where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

### 4.2 Preparation of Polynucleotides

A naturally occurring HKalpha polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated HKalpha polynucleotides. For example, restriction enzymes and probes can be used to isolate polynucleotide fragments, which comprise HKalpha nucleotide sequences. Isolated polynucleotides are in preparations which are free or at least 70, 80, or 90% free of other molecules.

HKalpha DNA molecules can be made with standard molecular biology techniques, using HKalpha mRNA as a template. HKalpha DNA molecules can thereafter be replicated using molecular biology techniques known in the art and disclosed in manuals such as Sambrook et al. (1989). An amplification technique, such as PCR, can be used to obtain additional copies of polynucleotides of the invention. The inventors have successfully demonstrated this approach.

Alternatively, synthetic chemistry techniques can be used to synthesize HKalpha polynucleotides. The degeneracy of the genetic code allows alternate nucleotide sequences to be synthesized which will encode a HKalpha polypeptide or a biologically active variant thereof.

### 4.3 Expression of Polynucleotides

To express a HKalpha polynucleotide, the polynucleotide can be inserted into an expression vector which contains the necessary elements for the transcription and translation of the inserted coding sequence. Methods which are well known to those skilled in the art can be used to construct expression vectors containing sequences encoding HKalpha polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a HKalpha enzyme polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those nontranslated regions of the vector enhancers, promoters, 5′ and 3′ untranslated regions which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORTI plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a HKalpha polypeptide, vectors based on SV40 or EBV can be used with an appropriate selectable marker.

### 5. Host Cells

According to certain embodiments of the subject invention, a HKalpha polynucleotide will need to be inserted into a host cell, for expression, processing and/or screening. A host cell strain can be chosen for its ability to modulate the expression of the inserted sequences or to process the expressed HKalpha polypeptide in the desired fashion. Such modifications of the polypeptide include, but are not limited to, acetylation, carboxylation, glycosylation, phosphorylation, lipidation, and acylation. Posttranslational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

Stable expression is preferred for long-term, high yield production of recombinant proteins. For example, cell lines which stably express HKalpha polypeptides can be transformed using expression vectors which can contain viral origins of replication and/or endogenous expression elements and a selectable marker gene on the same or on a separate vector. Following the introduction of the vector, cells can be allowed to grow for 12 days in an enriched medium before they are switched to a selective medium. The purpose of the selectable marker is to confer resistance to selection, and its presence allows growth and recovery of cells which successfully express the introduced HKalpha sequences. Resistant clones of stably transformed cells can be proliferated using tissue culture techniques appropriate to the cell type. See, for example, ANIMAL CELL CULTURE, R. I. Freshney, ed., 1986.

### 5.1 Detecting Expression

A variety of protocols for detecting and measuring the expression of a HKalpha polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a HKalpha polypeptide can be used, or a competitive binding assay can be employed. These and other assays are described in Hampton et al, SEROLOGICAL METHODS: A LABORATORY MANUAL, APS Press, St. Paul, Minn., 1990) and Maddox et al., J. Exp. Med. 158, 12111216, 1983).

### 5.2 Expression and Purification of Polypeptides

Host cells transformed with nucleotide sequences encoding HKalpha polypeptide can be cultured under conditions suitable for the expression and recovery of the protein from cell culture. The polypeptide produced by a transformed cell can be secreted or contained intracellular depending on the sequence and/or the vector used. As will be understood by those of skill in the art, expression vectors containing polynucleotides which encode HKalpha polypeptides can be designed to contain signal sequences which direct secretion of soluble HKalpha polypeptides through a prokaryotic or eukaryotic cell membrane or which direct the membrane insertion of membrane-bound HKalpha polypeptide.

### 6. Antibodies

Antibodies are referenced herein and various aspects of the subject invention utilize antibodies specific to HKalpha polypeptide(s). As described above, one example of a therapeutic agent for inhibiting HKalpha may pertain to an antibody. Any type of antibody known in the art can be generated to bind specifically to an epitope of a HKalpha polypeptide. The term “antibody” as used herein includes intact immunoglobulin molecules, as well as fragments thereof, such as Fab, F(ab′) 2, and Fv, which are capable of binding an epitope of a HKalpha polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a HKalpha polypeptide can be used therapeutically, as mentioned, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen. Antibodies useful for embodiments of the subject invention may be polyclonal, but are preferably monoclonal antibodies.

### 7. Ribozymes

Ribozymes may be one category of test compounds potentially useful as therapeutic agents for modulating HKalpha activity. Ribozymes are RNA molecules with catalytic activity. See, e.g., Cech, Science 236, 15321539; 1987; Cech, Ann. Rev. Biochem. 59, 543568; 1990, Cech, Curr. Opin. Struct. Biol. 2, 605609; 1992, Couture & Stinchcomb, Trends Genet. 12, 510515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

Accordingly, another aspect of the invention pertains to using the coding sequence of a HKalpha polynucleotide to generate ribozymes which will specifically bind to mRNA transcribed from the HKalpha polynucleotide. Methods of designing and constructing ribozymes which can cleave other RNA molecules in trans in a highly sequence specific manner have been developed and described in the art (see Haseloff et al. Nature 334, 585591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a HKalpha RNA target can be identified by scanning the target molecule for ribozyme cleavage sites. Once identified, short RNA sequences of between 15 and 20 ribonucleotides corresponding to the region of the target RNA containing the cleavage site can be evaluated for secondary structural features which may render the target inoperable. Suitability of candidate HKalpha RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease HKalpha expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells. Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

### 8. Interfering Molecules

HKalpha can be inhibited by a number of means including silencing via miRNA, shRNA, or siRNA, for example, directed to a portion of the sequence described at the genbank accession numbers provided herein. In one embodiment, the HKalpha inhibitor comprises an interfering molecule, and wherein the interfering molecule comprises a member selected from the group consisting of a phosphothioate morpholino oligomer (PMO), miRNA, siRNA, methylated siRNA, treated siRNAs, shRNA, antisense RNA, a dicer-substrate 27-mer duplex, and combinations thereof.

siRNA molecules can be prepared against a portion of a nucleotide sequence encoding HKalpha according to the techniques provided in U.S Patent Publication 20060110440, incorporated by reference herein, and used as therapeutic compounds. shRNA constructs are typically made from one of three possible methods; (i) annealed complementary oligonucleotides, (ii) promoter based PCR or (iii) primer extension. See Design and cloning strategies for constructing shRNA expression vectors, Glen J McIntyre, Gregory C FanningBMC Biotechnology 2006, 6:1 (5 Jan. 2006).

For background information on the preparation of miRNA molecules, see e.g. U.S. patent applications 20110020816, 2007/0099196; 2007/0099193; 2007/0009915; 2006/0130176; 2005/0277139; 2005/0075492; and 2004/0053411, the disclosures of which are hereby incorporated by reference herein. See also, U.S. Pat. Nos. 7,056,704 and 7,078,196 (preparation of miRNA molecules), incorporated by reference herein. Synthetic miRNAs are described in Vatolin, et al 2006 J Mol Biol 358, 983-6 and Tsuda, et al 2005 Int J Oncol 27, 1299-306, incorporated by reference herein. See also U.S. patent applications 20120034236 and 20110251255, incorporated by reference herein for further examples of interfering molecules for targeting HKalpha expression, for example.

### Other Modulators and High Throughput Techniques

### A. Modulators

The compounds tested as modulators of HKalpha modulation can be any small chemical compound, or a biological entity, such as a protein, sugar, nucleic acid or lipid. Typically, test compounds will be small chemical molecules and peptides. Essentially any chemical compound can be used as a potential modulator in the assays of the invention. The compounds can be dissolved in aqueous or organic solutions (e.g., methanol, DMSO, or a mixture of organic solvents). The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### B. Solid State and Soluble High Throughput Assays

In one embodiment the invention provides in vitro soluble assays in a high throughput format. In another embodiment, the invention provides soluble or solid phase based in vivo assays in a high throughput format, where the cell or tissue is attached to a solid phase substrate. Optionally, the in vitro assay is a solid phase assay.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule or cell of interest can be bound to the solid state component, directly or indirectly, via covalent or non covalent linkage of a tag and or a tag binder. A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochem ice's 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Labels and Means of Detection

Detectable labels and moieties can be primary labels (where the label comprises an element which is detected directly or which produces a directly detectable element) or secondary labels (where the detected label binds to a primary label, e.g., as is common in immunological labeling). An introduction to labels, labeling procedures and detection of labels is found in Polak & Van Noorden (1997) Introduction to Immunocytochemistry (2^{nd} ed. 1977) and Handbook of Fluorescent Probes and Research Chemicals, a combined handbook and catalogue Published by Molecular Probes, Inc., Eugene, Oreg. Primary and secondary labels can include undetected elements as well as detected elements.

The particular label or detectable group used in the assay is not a critical aspect of the invention, as long as it does not significantly interfere with the specific binding of an agent used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means.

Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³²P, ³³P, etc.), enzymes (e.g., horseradish peroxidase, alkaline phosphatase etc.), spectral colorimetric labels such as colloidal gold or colored glass or plastic (e.g. polystyrene, polypropylene, latex, etc.) beads.

The label may be coupled directly or indirectly to a component of the detection assay according to methods well known in the art. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. As indicated above, a wide variety of labels may be used, with the choice of label depending on sensitivity required, ease of conjugation with the compound, stability requirements, available instrumentation, and disposal provisions.

In general, a detector which monitors a particular probe or probe combination is used to detect the recognition reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling nucleic acids is digitized for subsequent computer analysis.

Preferred labels include those which utilize enzymes such as hydrolases, particularly phosphatases, kinases, esterases and glycosidases, or oxidotases, particularly peroxidases; chemiluminescence (e.g., enzymes such as horseradish peroxidase or alkaline phosphatase with substrates that produce photons as breakdown products; kits available, e.g., from Molecular Probes, Amersham, Boehringer-Mannheim, and Life Technologies/Gibco BRL); color production (using, e.g., horseradish peroxidase, β-galactosidase, or alkaline phosphatas

### EXAMPLES

### Example 1

### DOCP Caused Disturbances in Na⁺, K⁺, and Acid-Base Homeostasis

In the present study, desoxycorticosterone pivalate (DOCP) was used as a model of chronic mineralocorticoid excess.¹² DOCP has long-lasting effects resulting from esterase cleavage in the muscle to the active mineralocorticoid, desoxycorticosterone acetate.^{13, 14} The timings of DOCP-induced disturbances in body weight, Na⁺, K⁺, and acid-base homeostasis were determined and correlated with renal H⁺,K⁺-ATPase activity and H⁺,K⁺-ATPase α subunit expression. Disturbances in Na⁺, K⁺, Cl⁻, and HCO₃⁻ homeostasis were evident in DOCP-treated wild type mice after eight days. DOCP treatment also increased renal H⁺,K⁺-ATPase activity and mRNA expression for HKα₂ by this time point. This study also examined the physiological role of the HKα₁ and HKα₂ H⁺,K⁺-ATPases in mineralocorticoid-induced electrolyte and acid-base disturbances using mice that have disruption of either the HKα₁ gene (HKα₁^{-/-}) or both the HKα₁ and HKα₂ genes (HKα_{1,2}^{-/-}). The present studies reveal that the H⁺,K⁺-ATPases exert a profound influence on mineralocorticoid-mediated changes in Na⁺, K⁺, and acid-base homeostasis.

A primary goal of the study was to characterize the temporal changes in body weight, Na⁺, K⁺, and acid-base homeostasis during chronic mineralocorticoid excess. Body weight and blood electrolytes were measured over an eight day time course in untreated (control) mice and those treated with DOCP (1.7 mg) (Table 1). Excess body weight gain was apparent in DOCP-treated mice. DOCP treatment caused a considerable increase in body weight by day four but control mice exhibited no significant change in body weight over this time period (data not shown). The observed body weight gain in DOCP-treated mice is consistent with the known effect of DOCP to enhance Na⁺ and fluid volume retention. By the fourth day, DOCP treatment resulted in hypernatremia, an effect that started to wane by eight days. Moreover, DOCP treatment resulted in a reduction in blood [K⁺] by six days after DOCP administration Eight days of DOCP treatment also significantly increased blood [HCO₃⁻] in wild-type mice. The timing and magnitude of blood [HCO₃⁻] increases with DOCP treatment were reflected in a reciprocal decrease in blood [Cl⁻] by approximately 7 mM.

### Example 2

### DOCP Increased H⁺,K⁺-ATPase Activity in the Collecting Duct

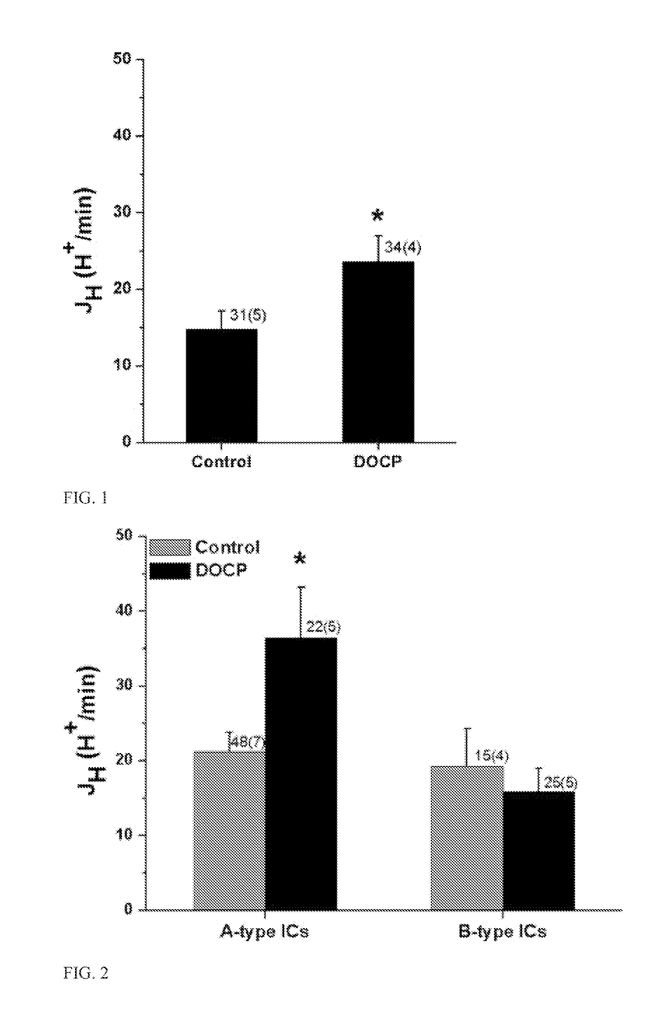

The results of the physiology studies showed that DOCP treatment led to the development of a reduced blood [K⁺] and greater blood [HCO₃⁻] within eight days. One likely mechanism for mineralocorticoid-induced changes in acid-base homeostasis was stimulation of renal H⁺,K⁺-ATPase activity in response to mineralocorticoid-induced K⁺ deficits. Therefore, H⁺,K⁺-ATPase activity was assessed in inner cortical collecting ducts from control and DOCP-treated mice. Individual intercalated cells from microperfused collecting ducts were loaded with the pH sensitive fluorescent dye, BCECF-AM. The addition of inhibitors of the Na⁺/H⁺ exchanger and the H⁺-ATPase allowed for the measurement of H⁺,K⁺-ATPase-mediated H⁺ secretion (J_{H}) in response to an acute intracellular acid load (NH₄Cl). The overall rate of H⁺,K⁺-ATPase-mediated J_{H} in intercalated cells from control mice was quite slow (˜15H⁺/min) (FIG. 1). However, a greater than 60% increase in H⁺,K⁺-ATPase-mediated J_{H} was observed in intercalated cells from DOCP-treated mice.

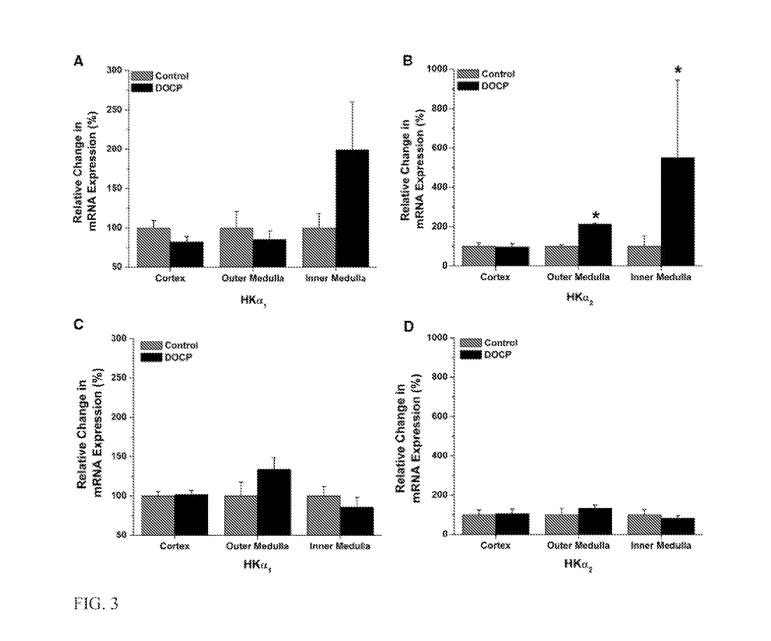

Additional experiments were conducted to identify which intercalated cell subtype was responsible for the DOCP-induced increase in H⁺,K⁺-ATPase mediated J_{H}. Again, the rate of H⁺,K⁺-ATPase-mediated J_{H} was measured in intercalated cells of inner cortical collecting ducts from control and DOCP-treated mice. At the end of the experiment, A- and B-type ICs were differentiated by peritubular Cl⁻ removal and return as has been described previously^{12,13} The characteristic inverse responses of A- and B-type intercalated cells to Cl⁻ removal and return were observed (Table 2). Intrinsic buffering capacity (βᵢ) was not significantly affected by DOCP treatment. There was no difference in the rate of H⁺,K⁺-ATPase mediated J_{H} in A- and B-type intercalated cells from control mice (FIG. 2). Although the rate of H⁺,K⁺-ATPase-mediated J_{H} was not affected in B type ICs, DOCP treatment increased H⁺,K⁺-ATPase-mediated J_{H} in A-type intercalated cells by approximately 60%.

### Example 3

### DOCP Induced Medullary HKα₂ mRNA Expression

The principal mechanism of mineralocorticoid action is through modulation of target gene transcription.¹⁵ Therefore, the next experiments evaluated the effect of DOCP on renal HKα₁ and HKα₂ mRNA expression. Real time quantitative PCR was used to investigate changes in steady state mRNA levels of H⁺,K⁺-ATPase α subunits, HKα₁ and HKα₂, in cortex, outer medulla, and inner medulla of control and DOCP-treated mice. Eight days after DOCP treatment, HKα₁ mRNA expression in all three kidney segments was not significantly altered compared to control (FIG. 3A). DOCP treatment had a tendency to increase HKα, expression in the inner medulla. DOCP did not affect HKα₂ mRNA expression in the cortex (FIG. 3B). However, HKα₂ mRNA levels were increased more than two-fold in the outer medulla and as much as five-fold in the inner medulla of DOCP-treated mice.

Deficits in K⁺ homeostasis have long been known to stimulate H⁺,K⁺-ATPase activity and HKα subunit expression. Therefore, the next experiments investigated whether the decreases in blood [K⁺] observed in DOCP-treated mice were responsible for the stimulation of HKα₂ mRNA expression. For this experiment, mice were fed a high K⁺ diet and then left untreated (control) or given DOCP treatment. Body weight and blood electrolytes were measured eight days after treatment and kidneys were collected for analysis of HKα, and HKα₂ mRNA expression by real time PCR. A high K⁺ diet abrogated the effect of DOCP on body weight gain, blood [K⁺], [Cl⁻], and [HCO₃⁻] in mice (Table 3). Similarly, the stimulation of HKα, or HKα₂ mRNA expression with DOCP was absent in mice fed a high K⁺ diet (FIGS. 3C and 3D). These results demonstrate that the stimulation of medullary H⁺,K⁺-ATPase α subunit mRNA expression with DOCP treatment is dependent on dietary K⁺ intake.

### Example 4

### HKα Null Mice Showed Altered Na⁺, K⁺, and Acid-Base Homeostasis with DOCP Treatment

The final set of experiments considered the physiological function of renal H⁺,K⁺-ATPases in the response to chronic mineralocorticoid excess and specifically characterized the effect of DOCP treatment on the electrolyte and acid-base homeostasis of mice with disruption of the H⁺,K⁺-ATPase HKα, subunit (HKα₁^{-/-}) or both the HKα, and HKα₂ subunits (HKα_{1,2}^{-/-}. Body weight change (%) and blood electrolytes were first measured in untreated wild type and knockout mice (Table 4). No appreciable body weight change (%) was observed in untreated mice of any genotype over eight days (data not shown). Blood [K⁺] was paradoxically greater in the HKα_{1,2}^{-/-} compared to wild type or the HKα₁^{-/-} mice. Blood [Cl⁻] was less in the HKα₁^{-/-} compared to either the wild type or HKα_{1,2}^{-/-} mice.

In a separate study, the effect of DOCP treatment of body weight change, blood electrolytes, and urinary and fecal electrolyte excretion was examined in wild type, HKα₁^{-/-}, and HKα_{1,2}^{-/-} mice on a normal diet. DOCP-induced body weight gain was comparable in wild type and HKα_{1,2}^{-/-} mice but was augmented nearly two-fold in the HKα₁^{-/-} mice (FIG. 4A). Blood [Na⁺] was similar in mice from all three genotypes (FIG. 4B). Although DOCP decreased blood [K⁺] in mice from all three genotypes, HKα_{1,2}^{-/-} mice still exhibited greater blood [K⁺] than wild type or HKα₁^{-/-} mice (FIG. 4C). The effect of DOCP to decrease blood [Cl⁻] (FIG. 4D) and increase blood [HCO₃⁻] (FIG. 4E) was eliminated in HKα_{1,2}^{-/-} mice.

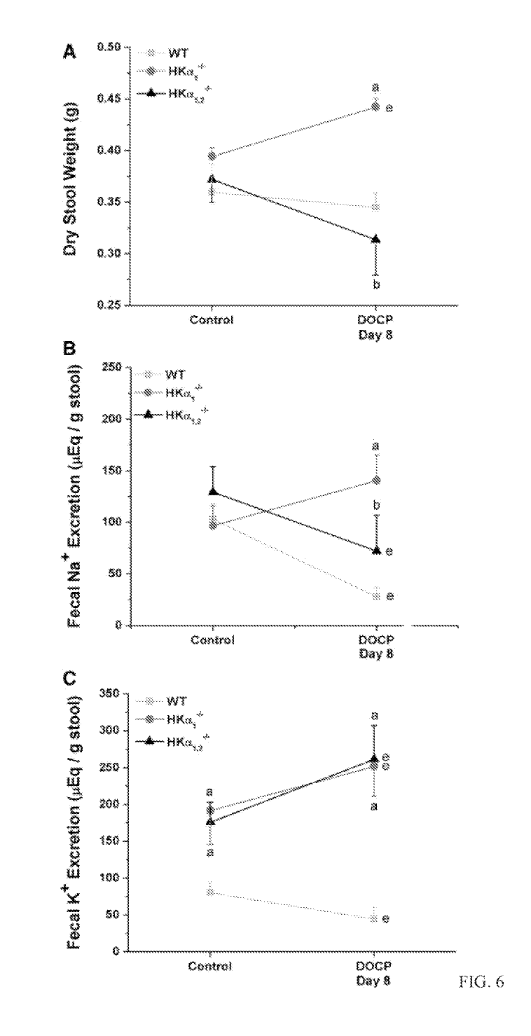

In order to more fully understand the mechanism for the observed differences in body weight gain and blood electrolytes between wild type and the HKα knockout mice, urinary and fecal Na⁺ and K⁺ excretion were measured the day before and eight days after DOCP treatment. Urine volume doubled by the end of DOCP treatment in both wild type and HKα_{1,2}^{-/-} mice but considerably decreased in HKα₁^{-/-} mice (FIGS. 5A and 5D). Over the course of DOCP treatment, HKα_{1,2}^{-/-} retained significantly less urinary Na⁺ than wild type or HKα₁^{-/-} mice (FIG. 5B). In comparison of day eight of DOCP treatment to control urinary Na⁺ retention, HKα₁^{-/-} mice retained more urinary Na⁺ than wild type mice at this time point as well (FIG. 5E). At eight days of DOCP treatment, urinary K⁺ retention was greater in HKα, mice than either wild type or HKα_{1,2}^{-/-} mice (FIGS. 5C and 5F).

Analysis of stool samples from wild type, HKα₁^{-/-}, and HKα_{1,2}^{-/-} mice revealed that HKα₁^{-/-} mice excreted 50% more dry stool weight than either the wild type or HKα_{1,2}^{-/-} mice when treated with DOCP, even though the mice were pair fed (FIG. 6A). Fecal Na⁺ excretion significantly decreased in DOCP-treated wild type and HKα_{1,2}^{-/-} mice and was significantly greater in DOCP-treated HKα_{1,}^{-/-} mice than wild type or HKα_{1,2}^{-/-} mice (FIG. 6B). Interestingly, fecal K⁺ loss was evident in both HKα₁^{-/-} and HKα_{1,2}^{-/-} mice in the control period and this loss increased ˜50% with DOCP treatment (FIG. 6C).

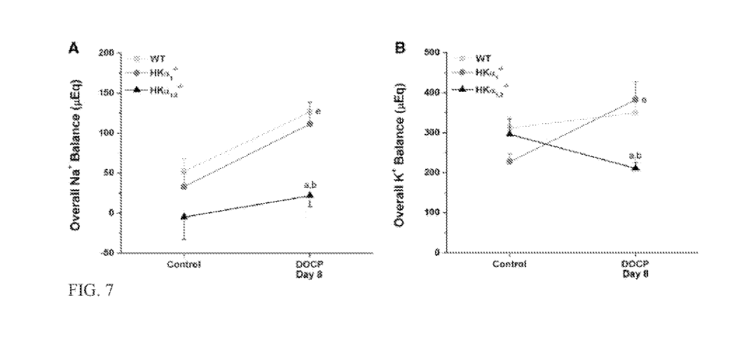

In the context of whole animal physiology, it is important to examine the overall electrolyte balance as the product of urinary and fecal excretion subtracted from the intake of that electrolyte. Overall Na⁺ and K⁺ balance were not significantly different in mice from any genotype under control conditions (FIG. 7). As expected, overall Na⁺ balance increased in wild type mice with DOCP treatment (FIG. 7A). Overall Na⁺ retention was significantly less in HKα_{1,2}^{-/-} mice than wild type or HKα₁^{-/-} mice on the eighth day of DOCP treatment. Also unlike HKα₁^{-/-} mice, HKα_{1,2}^{-/-} mice exhibited ˜40% less K⁺ retention than wild type mice with DOCP treatment (FIG. 7B). These results demonstrate conclusively that disruption of H⁺,K⁺-ATPases in mice abolishes the effect of DOCP to increase total body Na⁺ and K⁺ retention.

Discussion

In the present report, definitive evidence is provided showing that mineralocorticoids regulate the activity and expression of renal H⁺,K⁺-ATPases. Prolonged exposure to DOCP increased H⁺ secretion in the collecting duct via renal H⁺,K⁺-ATPases. More specifically, the increase in HKα₂ expression with DOCP treatment supports a particularly-important role of the HKα₂ H⁺,K⁺-ATPase isoform in mineralocorticoid-induced H⁺ secretion. Whole animal studies in HKα₁^{-/-} and HKα_{1,2}^{-/-} mice subjected to DOCP treatment highlight the significance of H⁺,K⁺-ATPases to mineralocorticoid-mediated disturbances in K⁺ and acid-base homeostasis as well as Na⁺ homeostasis. The overall deficit in K⁺ retention and lower blood [HCO₃] observed in DOCP-treated HKα_{1,2}^{-/-} mice signifies the importance of H⁺,K⁺-ATPases to facilitate K⁺ reabsorption and H⁺ secretion with mineralocorticoid excess. Importantly, the elimination of DOCP-induced Na⁺ retention in HKα_{1,2}^{-/-} mice and not in HKα₁^{-/-} mice indicates that the HKα₂-containing H⁺,K⁺-ATPases indirectly regulate mineralocorticoid-mediated Na⁺ retention which may present major implications for blood pressure regulation.

The effect of long term mineralocorticoid excess to change H⁺,K⁺-ATPase activity and its relation to dietary K⁺ intake has been previously examined by Eiam-Ong and colleagues.¹¹ In contrast to the results described in this paper, seven days exposure to low, normal, or high aldosterone levels in rats fed diets consisting of low, normal and high K⁺ content, did not significantly affect Schering 28080-sensitive (an H⁺,K⁺-ATPase inhibitor) ATPase activity in microdissected cortical and medullary collecting ducts. However, subsequent studies revealed that Schering 28080 was found to only inhibit the HKα₁-containing H⁺,K⁺-ATPases. The observed stimulation of H⁺,K⁺-ATPase activity in A-type intercalated cells of inner cortical collecting ducts from DOCP-treated mice coupled with the dramatic augmentation of HKα₂ mRNA expression in the renal medulla of DOCP-treated mice reveals that mineralocorticoids, in fact, do regulate renal H⁺,K⁺-ATPases.

The renal H⁺,K⁺-ATPases have been purported to act as primarily K⁺ reabsorptive mechanisms. Since hypokalemia has been shown to substantially increase medullary HKα₂ expression¹⁶⁻¹⁸ and hypokalemia was quite evident with DOCP treatment, the induction of HKα₂ expression in the outer and inner medulla of DOCP-treated mice could be a result of hypokalemia. Abolishment of DOCP-induced HKα₂ mRNA expression in the medulla by a high K⁺ is consistent with this hypothesis.

The increase in H⁺,K⁺-ATPase-mediated H⁺ secretion in the collecting duct and HKα₂ subunit expression in the kidney coincided with the development of a greater blood [HCO₃] in DOCP-treated wild type mice. The similar time course of these two events suggests that H⁺,K⁺-ATPases are responsible for a significant portion of the increase in blood [HCO₃⁻] with mineralocorticoid excess. Most importantly, DOCP treatment did not significantly increase blood [HCO₃⁻] in HKα_{1,2}^{-/-} mice. Taken together with the response of the wild type mice, these data strongly support the hypothesis that the H⁺,K⁺-ATPases mediate the development of mineralocorticoid-induced alkalosis.

Excessive body weight gain and urinary Na⁺ retention in DOCP-treated HKα₁^{-/-} mice and its elimination in HKα_{1,2}^{-/-} mice demonstrates that the mineralocorticoid-sensitive component of Na⁺ and fluid reabsorption depends directly or indirectly on the HKα₂-containing H⁺,K⁺-ATPases. Precedent for such a conclusion is supported by two separate sets of observations: 1) Both the HKα₁ and HKα₂ H⁺,K⁺-ATPases have been found to transport Na⁺ on the K⁺ binding site of the transporter;¹⁹⁻²¹ 2) evidence from Spicer et al²² supports a dependence of the epithelial Na⁺ channel on the HKα₂ H⁺,K⁺-ATPases. The HKα₂^{-/-} mice had reduced colonic epithelial Na⁺ channel activity on a normal diet that was exacerbated by dietary Na⁺ restriction. The observation that DOCP treatment affected urinary K⁺ retention in HKα₁^{-/-} and HKα_{1,2}^{-/-} mice as well as Na⁺ is more consistent with an indirect mechanism of H⁺,K⁺-ATPase-mediated Na⁺ reabsorption. The results of these studies provide evidence for an important role of the H⁺,K⁺-ATPases in normal K⁺ homeostasis and in mineralocorticoid-mediated effects on K⁺, acid-base, and Na⁺ balance. Future investigation into the mechanism(s) by which the renal H⁺,K⁺-ATPases contribute to Na⁺ and fluid balance promises to shed important light on the pathogenesis of mineralocorticoid hypertension.

Concise Methods for Examples 1-4

Animals

All animal use protocols were approved by the North Florida/South Georgia Veterans Administration Institutional Animal Care and Use Committee in accordance with the National Institute of Health Guide for the Care and Use of Laboratory Animals. Female C57BL/6J mice were purchased from Jax Labs (Bar Harbor, Me.) or bred in house. HKα₁^{-/-} and HKα_{1,2}^{-/-} mice were generous gifts from Dr. Gary Shull. The latter were generated from breeding HKα₁^{-/- 23} and HKα₂^{-/- 24} mice and backcrossing onto the C57BL/6J background strain. Genotypes were confirmed by PCR of genomic DNA as previously described.²⁵

Mice were fed normal lab chow, given free access to water, and given an i.m. injection of 1.7 mg DOCP (Percorten V, Novartis Pharmaceuticals). For perfusion and expression studies, mice were sacrificed with Na⁺ pentobarbital (i.p. 120 mg/kg) followed by cervical dislocation. For time course experiments, another group of animals were anesthetized with 3-4% isoflurane and aortic blood was collected into a heparinized syringe for immediate analysis of electrolytes and blood gases (Nova pHOx Plus analyzer, Nova Biomedical). For high K⁺ diet studies, mice were given a normal, powdered diet (TD99131, Teklad) supplemented with KCl to equal 5% K⁺ which was then made as a gel. Half of the mice were treated with DOCP. Blood and tissues were collected as in the time course experiments. For urinalysis experiments, mice were housed in metabolic cages for thirteen days and fed a normal gel diet with free access to a water bottle. On day five, mice were treated with DOCP as above. Urine and feces were collected daily. Urine electrolytes were measured on a clinical analyzer (Nova 16 analyzer, Nova Biomedical) and fecal electrolytes were measured on a digital flame photometer (Model 2655-00, Cole-Parmer).

Tubule Perfusion and pHᵢ Recovery

Inner cortical collecting ducts were hand dissected at 4° C., transferred to a mounting chamber and perfused as described previously.²⁵ The ratiometric, pH sensitive dye, BCECF-AM (15 μM, Invitrogen) was added to the luminal perfusate for 10 minutes and fluorescence measurements were performed after dye de-esterification. Tubules were equilibrated with 100 nM bafilomycin A₁ in the luminal perfusate and 10 μM EIPA in the peritubular solution for 20 minutes and inhibitors were present throughout the remainder of the experiment. Cells were acid loaded by a 3 minute, peritubular exposure to 40 mM NH₄Cl followed by NH₄Cl removal. Ratiometric intensity measurements 490/440) were made in individual, well defined ICs and converted to pH, using linear regression, based on a high K⁺/nigericin calibration curve. Recovery rates were calculated from the linear portion of the pH, recovery phase starting with the lowest pH, achieved. The intrinsic buffering capacity was calculated from the formula βᵢ=ΔHᵢ/ΔpHᵢ where ΔHᵢ is the change in the calculated [NH₄]ᵢ and ΔpHᵢ the change in pHᵢ during the acid loaded phase. Acid secretion rates (J_{H}) were calculated from the formula J_{H}=βᵢ*ΔpH, (U/min) and expressed as [H⁺]/min. A- and B-type ICs were differentiated using peritubular chloride removal and return in HCO₃⁻ containing solutions as described previously.²⁵⁻²⁷

Analysis of mRNA

RNA was recovered from cortex, medulla, and inner medulla with TRIzol reagent (Invitrogen). RNA was treated with DNase 1 (Ambion DNA Free) and converted to cDNA using SuperScript III (Invitrogen). For real time quantitative PCR, 20 ng cDNA was used to quantify mRNA expression with TaqMan Gene Expression (Applied Biosystems) primer/probe sets for HKα₁ (Atp4a, Mm00444423_m1), HKα₂ (Atp12a, Mm0131809_m1), and β-actin (Actb, Mm00607939_s1). Cycle threshold (Ct) values were normalized to β-actin, and relative expression (control set to 100%) was calculated by the ΔΔCt method.²⁸

Statistical Analyses

All data are represented as mean±standard error of the mean (SEM). Statistics were performed with Origin 8 and SigmaStat 3.1 and all graphs/plots were made with Origin 8. The effects of time and treatment or time and genotype were analyzed by a two-way repeated measures ANOVA with post-hoc Student-Newman-Keuls test. The effect of treatment duration or genotype were analyzed by one-way ANOVA with a post hoc Student-Newman-Keuls test. An unpaired Student's t-test was used to compare differences between control and treated groups. All P values less than 0.05 were considered significant.

### REFERENCES FOR EXAMPLES 1-4

1. Boscaro M, Ronconi V, Turchi F, Giacchetti G: Diagnosis and management of primary aldosteronism. Curr Opin Endocrinol Diabetes Obes, 15: 332-8, 2008.
2. Marney A M, Brown N J: Aldosterone and end-organ damage. Clin Sci (Lond), 113: 267-78, 2007.
3. Sowers J R, Whaley-Connell A, Epstein M: Narrative review: the emerging clinical implications of the role of aldosterone in the metabolic syndrome and resistant hypertension. Ann Intern Med, 150: 776-83, 2009.
4. Eaton D C, Malik B, Saxena N C, Al-Khalili O K, Yue G: Mechanisms of aldosterone's action on epithelial Na+ transport. J Membr Biol, 184: 313-9, 2001.
5. Kovacikova J, Winter C, Loffing-Cueni D, Loffing J, Finberg K E, Lifton R P, Hummler E, Rossier B, Wagner C A: The connecting tubule is the main site of the furosemide-induced urinary acidification by the vacuolar H+-ATPase. Kidney Int, 70: 1706-16, 2006.
6. Winter C, Schulz N, Giebisch G, Geibel J P, Wagner C A: Nongenomic stimulation of vacuolar H+-ATPases in intercalated renal tubule cells by aldosterone. Proc Natl Acad Sci USA, 101: 2636-41, 2004.
7. Gumz M L, Lynch I J, Greenlee M M, Cain B D, Wingo C S: The renal H+-K+-ATPases: physiology, regulation, and structure. Am J Physiol Renal Physiol, 298: F12-21, 2010.
8. Dos Santos P M, Freitas F P, Mendes J, Tararthuch A L, Fernandez R: Differential regulation of H+-ATPases in MDCK-C11 cells by aldosterone and vasopressin. Can J Physiol Pharmacol, 87: 653-65, 2009.
9. Gekle M, Silbernagl S, Oberleithner H: The mineralocorticoid aldosterone activates a proton conductance in cultured kidney cells. Am J Physiol, 273: C1673-8, 1997.
10. Jaisser F, Escoubet B, Coutry N, Eugene E, Bonvalet J P, Farman N: Differential regulation of putative K(+)-ATPase by low-K+ diet and corticosteroids in rat distal colon and kidney. Am J Physiol, 270: C679-87, 1996.
11. Eiam-Ong S, Kurtzman N A, Sabatini S: Regulation of collecting tubule adenosine triphosphatases by aldosterone and potassium. J Clin Invest, 91: 2385-92, 1993.
12. Kintzer P P, Peterson M E: Treatment and long-term follow-up of 205 dogs with hypoadrenocorticism. J Vet Intern Med, 11: 43-9, 1997.
13. Lynn R C, Feldman E C: Treatment of canine hypoadrenocorticism with microcrystalline desoxycorticosterone pivalate. Br Vet J, 147: 478-83, 1991.
14. Lynn R C, Feldman E C, Nelson R W: Efficacy of microcrystalline desoxycorticosterone pivalate for treatment of hypoadrenocorticism in dogs. DOCP Clinical Study Group. J Am Vet Med Assoc, 202: 392-6, 1993.
15. Fuller P J, Young M J: Mechanisms of mineralocorticoid action. Hypertension, 46: 1227-35, 2005.
16. Ahn K Y, Park K Y, Kim K K, Kone B C: Chronic hypokalemia enhances expression of the H(+)-K(+)-ATPase alpha 2-subunit gene in renal medulla. Am J Physiol, 271: F314-21, 1996.
17. Codina J, Delmas-Mata J T, DuBose T D, Jr.: Expression of HKalpha2 protein is increased selectively in renal medulla by chronic hypokalemia. Am J Physiol, 275: F433-40, 1998.
18. Nakamura S, Amlal H, Galla J H, Soleimani M: Colonic H+-K+-ATPase is induced and mediates increased HCO3− reabsorption in inner medullary collecting duct in potassium depletion. Kidney Int, 54: 1233-9, 1998.
19. Swarts H G, Klaassen C H, Schuurmans Stekhoven F M, De Pont J J: Sodium acts as a potassium analog on gastric H,K-ATPase. J Biol Chem, 270: 7890-5, 1995.
20. Swarts H G, Koenderink J B, Willems P H, De Pont J J: The human non-gastric H,K-ATPase has a different cation specificity than the rat enzyme. Biochim Biophys Acta, 1768: 580-9, 2007.
21. Zhou X, Xia S L, Wingo C S: Chloride transport by the rabbit cortical collecting duct: dependence on H,K-ATPase. J Am Soc Nephrol, 9: 2194-202, 1998.
22. Spicer Z, Clarke L L, Gawenis L R, Shull G E: Colonic H(+)-K(+)-ATPase in K(+) conservation and electrogenic Na(+) absorption during Na(+) restriction. Am J Physiol Gastrointest Liver Physiol, 281: G1369-77, 2001.
23. Spicer Z, Miller M L, Andringa A, Riddle T M, Duffy J J, Doetschman T, Shull G E: Stomachs of mice lacking the gastric H,K-ATPase alpha-subunit have achlorhydria, abnormal parietal cells, and ciliated metaplasia. J Biol Chem, 275: 21555-65, 2000.
24. Meneton P, Schultheis P J, Greeb J, Nieman M L, Liu L H, Clarke L L, Duffy J J, Doetschman T, Lorenz J N, Shull G E: Increased sensitivity to K+ deprivation in colonic H,K-ATPase-deficient mice. J Clin Invest, 101: 536-42, 1998.
25. Lynch I J, Rudin A, Xia S L, Stow L R, Shull G E, Weiner I D, Cain B D, Wingo C S: Impaired acid secretion in cortical collecting duct intercalated cells from H-K-ATPase-deficient mice: role of HKalpha isoforms. Am J Physiol Renal Physiol, 294: F621-7, 2008.
26. Lynch I J, Greenlee M M, Gumz M L, Rudin A, Xia S L, Wingo C S: Heterogeneity of H-K-ATPase-mediated acid secretion along the mouse collecting duct. Am J Physiol Renal Physiol, 298: F408-15, 2010.
27. Petrovic S, Spicer Z, Greeley T, Shull G E, Soleimani M: Novel Schering and ouabain-insensitive potassium-dependent proton secretion in the mouse cortical collecting duct. Am J Physiol Renal Physiol, 282: F133-43, 2002.
28. Livak K J, Schmittgen T D: Analysis of relative gene expression data using real-time quantitative PCR and the 2(−Delta Delta C(T)) Method. Methods, 25: 402-8, 2001.
J Am Soc Nephrol. 2001 December; 12(12):2554-64 is also cited for sequence information of HK alpha-2.
Kidney International (1999) 56, 1029-1036; is also cited for sequence information, and specifically highly conserved regions of HK alpha sequences. These sequences are non-limiting examples of sequences that can be targeted for therapeutical applications as well as screened against.

TABLE 1
Time course of the physiological effect of DOCP treatment in wild type mice.

| | Control (n) | Day 2 (n) | Day 4 (n) | Day 6 (n) | Day 8 (n) |
|---|---|---|---|---|---|
| Change in BW (%) | 0 ± 0 (7) | 0.06 ± 1.2 (7) | 2.0 ± 1.2 (7) | 5.4 ± 1.4 (7)**^{,†} | 6.1 ± 1.2 (7)**^{,†} |
| Blood [Na⁺] (mM) | 148 ± 0.41 (13) | 150 ± 1.3 (3) | 153 ± 1.8 (4)* | 153 ± 0.80 (3)* | 150 ± 0.41 (3) |
| Blood [K⁺] (mM) | 3.9 ± 0.08 (14) | 3.5 ± 0.12 (4) | 3.5 ± 0.17 (4) | 2.9 ± 0.21 (3)* | 2.8 ± 0.07 (3)*^{,†} |
| Blood [Cl⁻] (mM) | 118 ± 0.51 (13) | 120 ± 1.8 (3) | 116 ± 0.95 (4) | 117 ± 0.35 (3) | 111 ± 0.82 (3)*^{,}**^{,†,‡} |
| Blood [HCO₃⁻] (mM) | 18 ± 0.51 (14) | 19 ± 0.87 (4) | 20 ± 1.2 (4) | 20 ± 0.53 (3) | 23 ± 2.0 (3)* |

BW, body weight. Day signifies days of DOCP treatment.
*denotes significant difference from control,
**from day 2,
^{†}from day 4, and
^{‡}from day 6.

TABLE 2
Characterization of intercalated cell (IC) subtypes from inner CCD of control and DOCP-treated mice.

| | A-type IC | | B-type IC | |
|---|---|---|---|---|
| | Control (n = 48.7) | DOCP (n = 22.4) | Control (n = 15.5) | DOCP (n = 24.5) |
| Cl⁻ Removal (U/min) | 0.52 ± 0.04 | 0.59 ± 0.04 | −0.31 ± 0.06 | −0.35 ± 0.03 |
| Cl⁻ Return (U/min) | −0.39 ± 0.03 | −0.37 ± 0.06 | 0.37 ± 0.04 | 0.37 ± 0.03 |
| βᵢ (U/min) | 69.1 ± 6.91 | 86.8 ± 16.6 | 68.7 ± 10.5 | 87.7 ± 9.02 |

U/min, change in pH;
βᵢ, buffering capacity;
n = cells(tubules)

TABLE 3
Effect of high K⁺ (5%) diet on physiological response of wild
type mice to DOCP treatment

| | Control (n = 4) | DOCP (n = 4) |
|---|---|---|
| Body weight change (g) | −0.42 ± 0.09 | −0.17 ± 0.27 |
| Blood [Na⁺] (mM) | 149 ± 0.99 | 151 ± 0.88 |
| Blood [K⁺] (mM) | 4.25 ± 0.06 | 4.30 ± 0.18 |
| Blood [Cl⁻] (mM) | 119 ± 0.45 | 121 ± 1.30 |
| Blood [HCO₃⁻] (mM) | 17.3 ± 0.64 | 18.2 ± 1.01 |

TABLE 4
Differences in blood chemistry of wild type (WT) and HKα null mice

| | WT | HKα₁^{-/-} | HKα_{1,2}^{-/-} |
|---|---|---|---|
| | (n = 13-14) | (n = 5) | (n = 9 |
| Blood [Na⁺] (mM) | 148 ± 0.41 | 149 ± 1.1 | 148 ± 0.58 |
| Blood [K⁺] (mM) | 3.9 ± 0.08 | 3.9 ± 0.12 | 4.4 ± 0.17* |
| Blood [Cl⁻] (mM) | 118 ± 0.51 | 114 ± 1.3* | 116 ± 0.91 |
| Blood [HCO₃⁻] (mM) | 18 ± 0.51 | 21 ± 1.2 | 19 ± 0.69 |

*denotes significant difference from WT.

### Example 5

### Mediation of Mineralcorticoid Blood Pressure

### Introduction:

Over stimulation of the amiloride-sensitive epithelial Na channel (ENaC) in the principal cells of the collecting duct by aldosterone and insulin may be responsible for a large portion of hypertension in modern society (Bubien 2010 Review). H,K-ATPases can directly reabsorb Na in place of K and selective H,K-ATPase inhibitors have also been shown to decrease net Na reabsorption by the CD (Buffin Meyer, Swarts, wingo papers refs)

HKα2 null (HKα₂^{-/-}) mice exhibited greater fecal Na excretion than WT mice when fed a Na restricted diet and the HKα₂^{-/-}) displayed a reduction in colonic amiloride-sensitive short circuit current (Spicer)

Without being bound to any mechanistic theory, it is hypothesized that the renal H,K-ATPases are required for maximal Na reabsorption under DOCP-stimulated conditions. ENaC subunit mRNA and protein expression levels were measured in kidneys from WT and HKα_{1,2}^{-/-} mice and differences in food and water intake and urinary aldosterone levels. Radiotelemetry was performed to determine whether BP was different between WT and HKα_{1,2}^{-/-} mice under normal and DOCP-stimulated conditions.

### Methods

Animals.

All animal use was in compliance with the American Physiological Society's Guiding Principles in the Care and Use of Laboratory Animals, and animal use protocols were approved by the North Florida/South Georgia Veterans Administration Institutional Animal Care and Use Committee.

For analysis of renal ENaC expression, male C56BL/6 WT and C56BL/6 HKα_{1,2}^{-/-} mice (age range of 12-16 weeks) were fed a normal gel diet consisting of 45% powdered food (Teklad 99131, 0.2% Na, 0.5% K), 1% agar, and 54% deionized water for 8 days. At the end of the experiment, mice were sacrificed by exsanguination under 3-4% isoflurane anesthesia followed by cervical dislocation. Kidneys were dissected into cortex and medulla on a cold plate and stored at −80° C. for use in expression studies.

In another experiment, male C56BL/6 WT and C56BL/6 HKα_{1,2}^{-/-} mice (age range of 12-16 weeks) were placed in metabolic cages (Nalgene) for 7 days. One group of mice was fed the normal gel diet as described above ad libitum for 7 days and another group were pair fed the same diet for 7 days. Daily body weight measurements were performed and urine collected every 24 hr. Urinary aldosterone levels (Aldosterone EIA, Cayman Chemical) were measured in 24 hr urine collections.

Chemicals.

All chemicals were obtained from Sigma or Fisher Scientific.

Quantitative Real Time PCR.

TRIzol® reagent (Invitrogen) was used to extract RNA from renal cortex and medulla according to the manufacturer's protocol. RNA samples were treated with DNase1 to remove DNA contaminants (DNA Free, Ambion). The RNA concentration and quality were determined by spectrophotometric analysis (absorbance 260 nm/280 nm from 1.9-2.0). RNA (1 or 2 μg) was converted to cDNA using SuperScript® III First Strand Synthesis SuperMix for quantitative reverse transcriptase (RT)-PCR (Invitrogen). TaqMan® Gene Expression Assays (Applied Biosystems) were used for quantitative PCR experiments. The primer/probe sets used include Scnn1a (Mm00803386_m1), Scnn1b (Mm00441215_m1), Scnn1g (Mm00441228_m1), and Actb (Mm00607939_s1). The 2× TaqMan® Universal PCR Mix-No AmpErase® Ung, a 20× primer/probe set (see above), and 20 ng cDNA were used in a 25 μL real time PCR reaction. Duplicate reactions were run in an Applied Biosystems 7500 Real Time PCR machine according to the manufacturer's instructions. The ΔΔ cycle threshold (Ct) method was used to determine relative expression. Ct values were normalized to the endogenous control gene, Actb. The ΔΔCt was calculated by subtraction of each individual ΔCt (experimental gene Ct-control gene Ct for each sample) from the average ΔCt of the control samples. Relative fold change in expression was calculated using 2^{-ΔΔCt}. The fold change for control samples (WT) is expressed as 1.

Western Blot Analysis.

Proteins (25 μg) were denatured at 95° C. in sample buffer (NuPage® LDS Sample Buffer, Invitrogen) containing 5% 2-mercaptoethanol. The protein samples and protein ladder (Dual Color Precision Plus Protein Standard, Bio-Rad) were loaded onto a 4-20% gradient tris-HCl polyacrilamide gel (Ready Gel, Bio-Rad) in Tris-glycine SDS buffer. The gels were run at 50 V until the ladder cleared the stacking gel then at 100-120 V until dye front reached the bottom of the gel. The proteins were transferred to polyvinylidene fluoride membranes at 50 V for 3 hr at 4° C. The transferred proteins were washed in tris-buffered saline (TBS) for 5 min then blocked in 2% Rodeo® blocker (USB) in TBS with 0.05% Saddle Soap® (USB) (TBS-S) for 1 hr at room temperature. The membrane was incubated overnight at 4° C. in primary antibody (1:1000 rabbit anti-αENaC or anti-γENaC) diluted in blocking buffer. The membrane was washed twice in TBS-S for 10 min each and incubated in secondary antibody (anti-rabbit IgG, USB) for 1 hr at room temperature. The membrane was washed again in TBS-S three times for 10 min each and rinsed in TBS. The membrane was incubated in Rodeo® ECL reagent (USB) for 5 min and signal detected by exposure to x-ray film. To reprobe the same blot with a different primary antibody, the blot was incubated in stripping solution (2% SDS, 100 mM 2-mercaptoethanol, 62.5 mM Tris (pH 6.7)) for 30 min at 70° C. The blots were washed in TBS twice for 5 min and then reblocked and probed as per above protocol.

To compare expression between genotypes, densitometric values for each band detected with each primary antibody were measured using the Un-Scan-It Gel Analysis software (Silk Scientific). The densitometric values obtained for a band detected with an experimental primary antibody (anti-αENaC and anti-γENaC) were divided by the values obtained for the loading control primary antibody (anti-6-actin) to correct for protein loading differences between samples. The percent differences in the corrected values were compared between WT (set at 100%) and knockout mice.

Radiotelemetry.

Age matched, male C56BL/6 WT and C56BL/6 HKα_{1,2}^{-/-} mice were anesthetized using 5% isoflurane, and radiotelemetry transmitters (PA-C10, Data Sciences, St. Paul, Minn.) were implanted under sterile conditions (1-3% continuous isoflurane). The catheter was placed in the left carotid artery and the housing of the transmitter was implanted subcutaneously on the animal's right flank. Mice were allowed to recover for a period of at least 14 days prior to recordings. These mice were not subject to metabolic cage experiments or blood and tissue collections. Mice were fed a normal lab chow (Teklad 2018, 0.2% Na and 0.5% K) and allowed free access to water. DOCP treated mice received an intramuscular injection of 1.7 mg DOCP (Percorten V, Novartis Pharmaceuticals) and were fed the normal gel diet described previously supplemented with NaCl to equal 0.4% Na⁺ ad libitum for 8 days with free access to water. Radiotelemetry recordings were performed one week before and one week after DOCP treatment. At the time of the control recording, mice ranged in age from 15-25 weeks. Recording were made for 2 minutes at the beginning of each hour or for 2 minutes every 10 minutes for 48 hours. Moving averages were calculated for each hour. Comparisons of systolic BP, HR, and LA were made for the main effects 1) DOCP treated versus untreated and 2) WT versus HKα_{1,2}^{-/-} mice. Differences between the daytime (06:00-17:59) and nighttime (18:00-05:59) values for these parameters were also compared.

Statistical Analyses.

The results are expressed as mean±standard error of the mean (SEM). Statistics were performed using the following software programs: Origin 7 (OriginLab Corp), Origin 8 (OriginLab Corp), or SigmaPlot 11 (Systat Software Inc). Paired or unpaired Student's t-Test, as appropriate, was used to compare two individual groups (i.e. genotype, treatment, etc.). Two way repeated measure ANOVA with post-hoc Student-Newman-Keuls test, if appropriate, was used to compare the main effects (i.e. time and treatment or genotype and treatment). Differences between groups were considered statistically significant at an a level of P<0.05.

### Results:

Renal ENaC mRNA and Protein Expression in WT and HKα_{1,2}^{-/-} Mice.

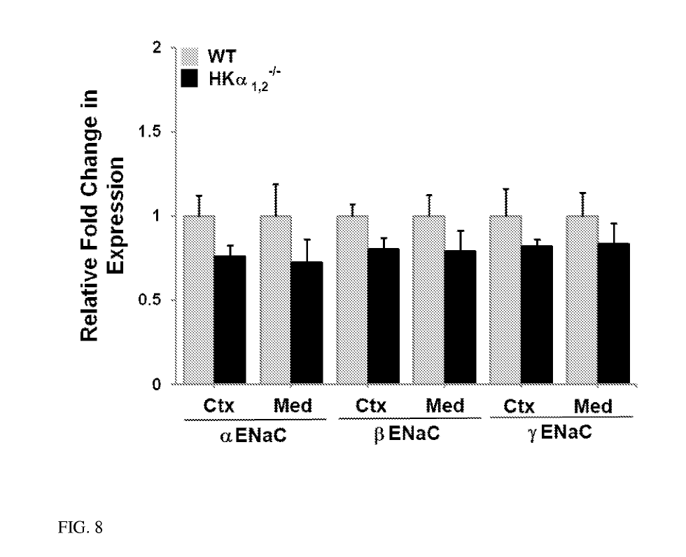

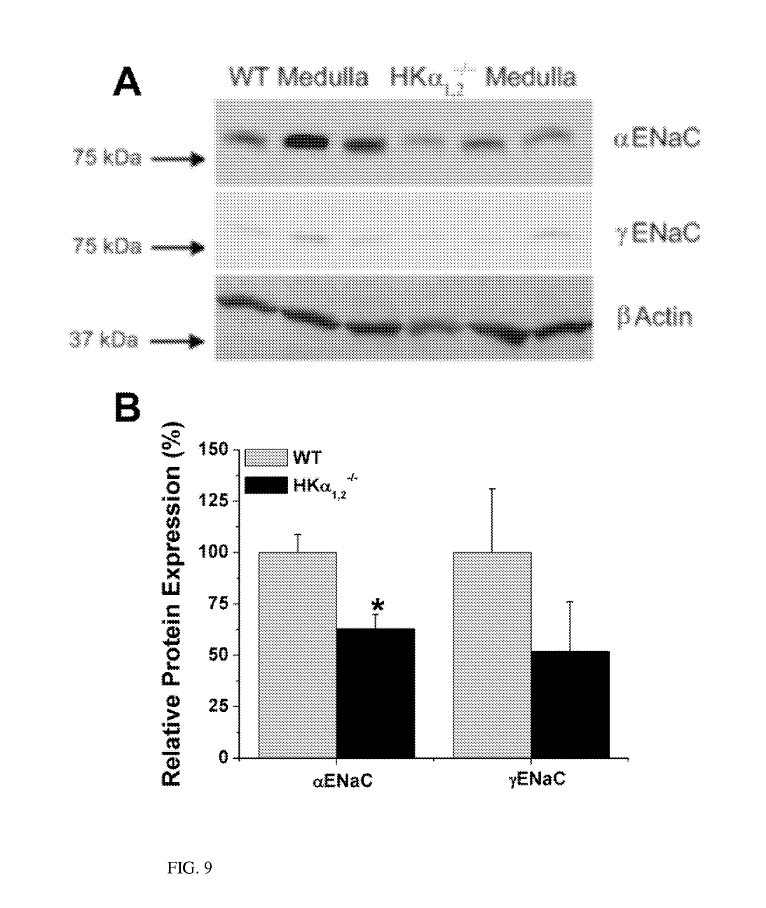

Real time PCR was performed to determine relative mRNA expression of α-, β-, and γENaC in renal cortex and medulla from male WT and HKα_{1,2}^{-/-} mice on a normal gel diet. mRNA expression of α-, β-, and γENaC subunits was not significantly different at the 0.05 level in the cortex and medulla of HKα_{1,2}^{-/-} mice compared to WT mice although all values were consistently less in HKα_{1,2}^{-/-} mice (FIG. 8). ENaC subunit total protein expression was also determined in renal medulla of WT and HKα_{1,2}^{-/-} mice by Western blot analysis. A representative Western blot is shown in FIG. 9A. A band was detected at the expected size for full length α- and γENaC protein (˜85 kDa). Medullary αENaC protein expression was clearly reduced in HKα_{1,2}^{-/-} mice compared to WT. Band densitometry revealed that HKα_{1,2}^{-/-} mice had ˜40% less αENaC protein expression than WT mice (FIG. 9B).

### Food Restriction and Urinary Aldosterone Levels in WT and HKα_{1,2}^{-/-} Mice.

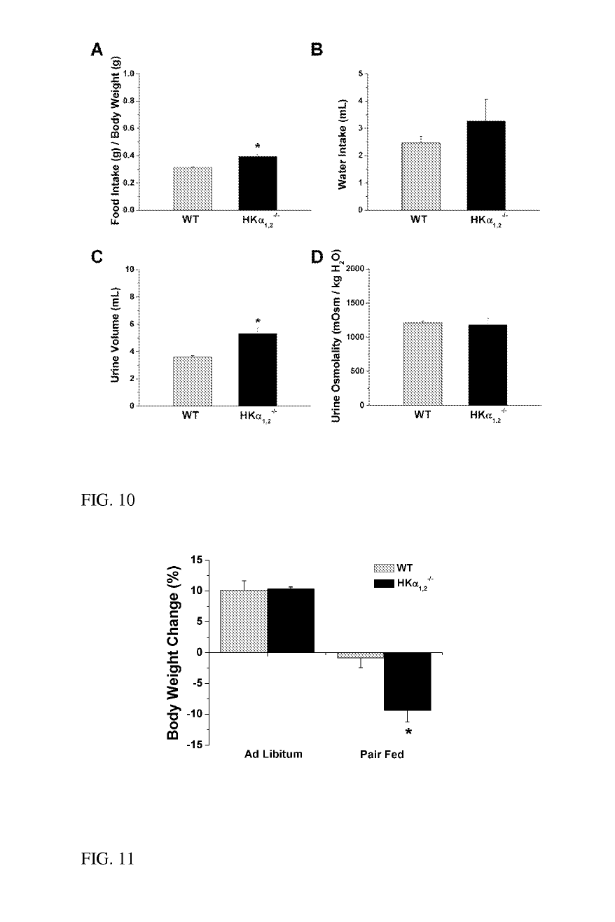

Previous experiments indicated that HKα_{1,2}^{-/-} mice have a tendency to consume more food than WT when fed ad libitum (data not shown). In light of these and our previous findings, it was hypothesized that the greater food intake was the result of impaired Na conservation. Therefore, this previous observation was examined by measuring food and H₂O intake in WT and HKα_{1,2}^{-/-} mice fed a normal gel diet ad libitum for one week. Urine volume and osmolality were also measured in WT and HKα_{1,2}^{-/-} mice. On an ad libitum diet, HKα_{1,2}^{-/-} mice consumed significantly more food per body weight (g) than WT mice despite no significant gain in body weight (FIG. 10A). When expressed as ml·(gm body weight)⁻¹·(24 h)⁻¹ there was a tendency for both H₂O intake and urine excretion to be slightly greater in HKα_{1,2}^{-/-} than WT mice (FIGS. 10B & 10C), and the absolute rate of urine excretion in ml·(24 h)⁻¹ was greater in double knockouts than WT mice (data not shown). Since the double knockouts consumed more gel food than WT mice, this implies a greater H₂O intake through the gel food. Consistent with this, urine osmolality was the same between the two genotypes (FIG. 10D).

Also examined was the ability of WT and HKα_{1,2}^{-/-} mice to maintain body weight during ad libitum and pair fed (food/Na⁺ restricted) conditions. WT and HKα_{1,2}^{-/-} mice were pair fed a normal gel diet or fed ad libitum with free access to H₂O for one week. Both WT and HKα_{1,2}^{-/-} mice gained a similar amount of body weight when fed ad libitum (FIG. 11). In contrast, feeding HKα_{1,2}^{-/-} mice the same amount of food as WT mice caused a considerable loss of body weight in the HKα_{1,2}^{-/-} mice. The body weight loss of HKα_{1,2}^{-/-} mice suggests an impairment in sodium conservation and a resultant loss of fluid volume.

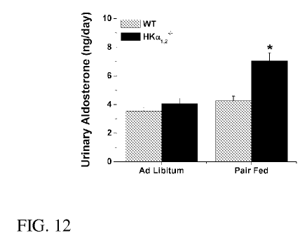

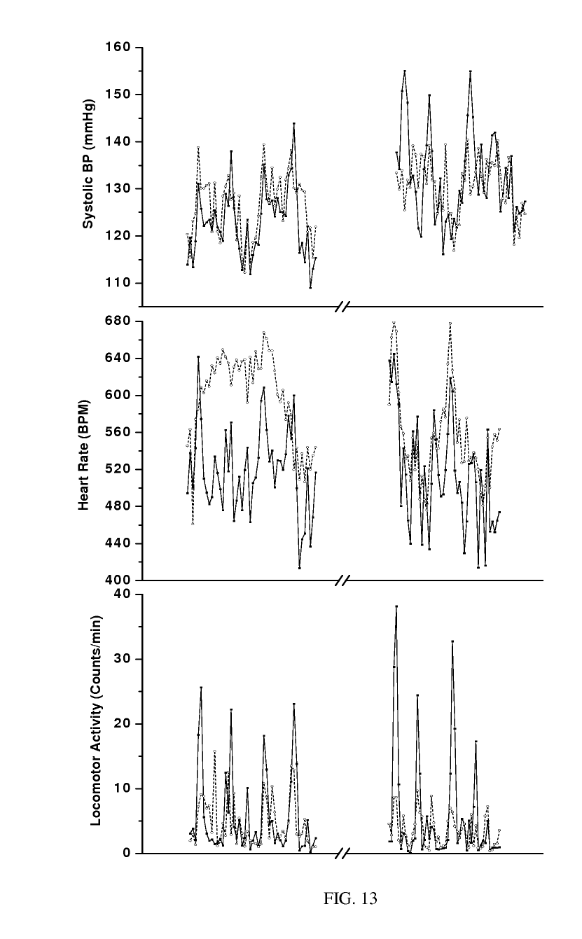

Dietary Na deficiency is known to increase plasma and urinary aldosterone levels (7). Therefore, urinary aldosterone levels in male WT and HKα_{1,2}^{-/-} mice fed ad libitum and in HKα_{1,2}^{-/-} mice pair fed to WT mice was measured. Urinary aldosterone excretion was similar between the genotypes during ad libitum intake (FIG. 12). However, pair feeding caused a pronounced increase in urinary aldosterone in HKα_{1,2}^{-/-} mice indicative of Na loss.

### Blood Pressure, Heart Rate, and Locomotor Activity in WT and HKα_{1,2}^{-/-} Mice.

Radiotelemetry was performed in order to assess genotypic differences in systolic BP, HR and LM during normal and DOCP treatment. Example 48 hr tracings of systolic BP, HR, and LA comparing WT and HKα_{1,2}^{-/-} during normal and DOCP treatment are shown in FIG. 13. MAP for WT and HKα_{1,2}^{-/-} are reported in Table 5.1. Both genotypes exhibited higher systolic BP at night during their most active phase compared with during the day when they are at rest. DOCP treatment significantly increased systolic BP during the day and night in the WT. However, DOCP treatment failed to significantly increase daytime or nighttime systolic BP in the HKα_{1,2}^{-/-} mice.

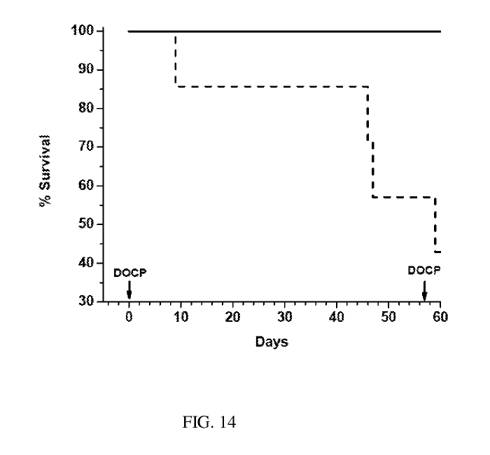

FIG. 14 shows the survival of nine WT and seven HKα_{1,2}^{-/-} mice over 60 days. These animals all received two injections of DOCP on day zero and day 57. Four of the HKα_{1,2}^{-/-} mice died at day's nine 46, 47, and 59 for an overall survival at 60 days of 57% for the HKα_{1,2}^{-/-} mice whereas there were no deaths in the nine WT animals X² (log-rank): 4.554 (P<0.05).

Heart rate (Table 5.2) was significantly faster during the night than during the day in both genotypes. During the day, the HR of HKα_{1,2}^{-/-} mice was significantly faster (˜10%) than the HR of the WT. A significant decrease in HR during the night was observed with DOCP treatment in the WT but not in the HKα_{1,2}^{-/-} mice.

Locomotor activity (Table 5.3) in both genotypes increased during the night; however, the HKα_{1,2}^{-/-} mice were less active than WT both during the day and night. DOCP treatment did not significantly affect LA.

TABLE 5.1
Mean arterial pressures (mmHg) in WT and HKα1,2−/− mice.

| | WT | HKα1,2−/− |
|---|---|---|
| | (n = 9) | (n = 7) |
| Daytime (Inactive) | | |
| Control | 105.0 ± 1.5 | 105.8 ± 1.7 |
| DOCP* | 112.9 ± 2.3** | 112.7 ± 1.8** |
| Nighttime (Active)† | | |
| Control | 113.2 ± 1.5 | 112.5 ± 2.3 |
| DOCP* | 122.5 ± 2.9** | 118.5 ± 2.4 |

*P < 0.001 versus Control at same time of day (two-way repeated measure ANOVA).
**P < 0.02 versus Control during same time of day (Student's t-Test).
†P < 0.01 versus Daytime during Control or DOCP

TABLE 5.2
Heart rate (beats/min) in WT and HKα1,2−/− mice

| | WT | HKα1,2−/−** |
|---|---|---|
| | (n = 9) | (n = 7) |
| Daytime | | |
| Control | 508.9 ± 9.97 | 557.2 ± 24.5 |
| DOCP | 496.7 ± 17.5 | 554.8 ± 21.4 |
| Nighttime† | | |
| Control | 561.3 ± 13.0 | 595.5 ± 21.6 |
| DOCP | 533.5 ± 17.2 | 580.5 ± 20.1 |

**P < 0.05 versus WT during the daytime (two-way repeated measure ANOVA).
†P < 0.01 versus Daytime during Control or DOCP (two-way repeated measure ANOVA).

TABLE 5.3
Locomotor activity in WT, HKα1−/−− and HKα1,2−/− mice

| | WT | HKα1,2−/−** |
|---|---|---|
| | (n = 9) | (n = 7) |
| Daytime | | |
| Control | 4.732 ± 0.535 | 2.626 ± 0.389 |
| DOCP* | 3.913 ± 0.407 | 3.267 ± 0.603 |
| Nighttime† | | |
| Control | 8.492 ± 0.844 | 4.282 ± 0.380 |
| DOCP | 7.036 ± 0.735 | 5.410 ± 1.078 |

*P < 0.05 versus control at the same time of day (two-way repeated measure ANOVA).
**P < 0.05 versus WT during the Nighttime (two-way repeated measure ANOVA).
†P < 0.01 versus Daytime during Control phase (two-way repeated measure ANOVA).

### Example 6

### Dietary Potassium Depletion in H⁺,K⁺-ATPase α₁ and α₂ Subunit Knockout Mice: Effect on Potassium and Sodium Excretion

### Introduction

The transition from a hunter-gather to agrarian and industrialized society dramatically changed the primary food types consumed by humans (Adrogue and Madias, 2007; Frassetto et al., 2009). These modern diets consist of refined grains and sugars and substantially greater amounts of fats and salt than the hunter-gather diet of lean meats and vegetables. The increase in salt (sodium (Na) chloride) consumption comes at the expense of potassium (K⁺) salts which are abundant in fruits and vegetables. Over the past few decades, several studies have shown that dietary K⁺ deficiency stimulates renal Na⁺ retention thus promoting the development of hypertension and exacerbating pre-existing hypertension (Krishna et al., 1987; Krishna et al., 1989; Krishna and Kapoor, 1991; 1993; Coruzzi et al., 2001; Coruzzi et al., 2003). In a relatively recent study, researchers observed noteworthy improvements in blood pressure within 10 days of patients receiving a hunter-gather (paleolithic) type diet with a high K⁺/Na⁺ intake ratio (4.3) compared to those receiving a contemporary diet with a low K⁺/Na⁺ ratio (0.6) (Frassetto et al., 2009). Investigation into the mechanisms responsible for maintaining K⁺ balance and eliciting the Na⁺ retention during low dietary K⁺ intake is essential for understanding the pathophysiology of dietary K⁺ deficiency.

For the better of two decades, H⁺,K⁺-ATPases have been viewed as the primary mechanisms for K⁺ reabsorption within the most distal portions of the kidney and gastrointestinal tract (Greenlee et al., 2010; Gumz et al., 2010). H⁺,K⁺-ATPases are composed of two subunits; the α subunit hydrolyzes ATP to exchange H⁺ and K⁺ across the plasma membrane while the β subunit controls membrane trafficking and degradation of the enzyme. Two genes encoding H⁺,K⁺-ATPase α subunits have been identified: Atp4a (HKα₁) and Atp12a (HKα₂). Atp4a was first described and has greatest expression in the stomach; Atp12a is most highly expressed in the distal colon. Both α subunits are expressed and active in the apical plasma membrane of intercalated cells of the renal collecting duct (Lynch et al., 2008; Lynch et al., 2010).

Several lines of evidence suggest a role for the H⁺,K⁺-ATPases in K⁺ conservation during dietary K⁺ restriction. First, low dietary K⁺ stimulates the activity of renal and colonic H⁺,K⁺-ATPases (Doucet and Marsy, 1987; Wingo, 1989; Sweiry and Binder, 1990). Second, in the kidney and colon, Atp12a (HKα₂) mRNA and protein expression dramatically increase with dietary K⁺ depletion (Ahn et al., 1996a; Codina et al., 1997; Kraut et al., 1997; Codina et al., 1998). One study has reported that renal cortical Atp4a (HKα₁) mRNA expression increases with dietary K⁺ depletion as well (Ahn et al., 1996b). Accordingly, HKα₂^{-/-} mice exhibited severe hypokalemia with dietary K⁺ restriction, confirming the importance of H⁺,K⁺-ATPases to the maintenance of K⁺ homeostasis. However, it was surprising that HKα₂^{-/-} mice only exhibited greater fecal (not urinary) K⁺ loss than WT. The authors interpreted that to mean the renal HKα₂-containing H⁺,K⁺-ATPases do not participate in net urinary K⁺ reabsorption during dietary K⁺ restriction (Meneton et al., 1998). However, it is conceivable that the remaining HKα₁ subunit in the HKα₂^{-/-} mice compensated for the missing HKα₂ H⁺,K⁺-ATPase isoform. This compensation would produce no observable renal K⁺ handling problem in the HKα₂^{-/-} mice.

Therefore, the studies presented in this example investigated whether mice null for HKα₁ (HKα₁^{-/-}) or both HKα₁ and HKα₂ (HKα_{1,2}^{-/-}) exhibited deficits in urinary and fecal K⁺ excretion with removal of dietary K⁺. Additionally, these studies determined whether the HKα_{1,2}^{-/-} mice displayed changes in Na⁺ balance as was previously observed in the context of mineralocorticoid excess (Greenlee et al., 2011). Based on the results from that previous publication, it is reasonable that renal H⁺,K⁺-ATPases may be involved in the urinary Na⁺ retention observed during dietary K⁺ deficiency (Krishna et al., 1987; Ray et al., 2001).

### Materials and Methods

### Animals.

All animal use was approved by the North Florida/South Georgia Veteran's Administration Institutional Animal Care and Use Committee in accordance with the NIH Guide for the Care and Use of Laboratory Animals. C57BL/6J (WT) mice were purchased from Jackson Laboratories (Bar Harbor, Me.) or bred in house. The generation and genotyping of HKα₁^{-/-} and HKα_{1,2}^{-/-} mice has been previously described (Lynch et al., 2008). For long term K⁺ depletion experiments, age-matched male WT and HKα_{1,2}^{-/-} mice (12-16 weeks, n=6-8 per genotype and diet) were housed in metabolic cages (Nalgene) and given either a control (Teklad 99131; 0.6% K⁺, 0.2% Na⁺) or K⁺ deplete (Teklad 99134; 0% K⁺, 0.2% Na) gel diet consisting of 45% food, 1% agar, and 54% water for eight days. For early K⁺ depletion studies, male WT and HKα_{1,2}^{-/-} mice (12-16 weeks, n=4-5 per genotype) were housed in metabolic cages where they were pair fed the control gel diet for 4 days then switched to the K⁺ deplete gel diet for 4 days. To assess the effect of dietary potassium depletion on blood potassium in HKα₁^{-/-} mice, male WT and HKα₁^{-/-} mice (12-16 weeks, n=4 per genotype) were housed in their normal cages and pair fed the control gel diet for 3 days then switched to the K⁺ deplete gel diet for 8 days. At the end of each of these experiments, mice were anesthetized with 3-4% isoflurane and arterial blood was collected anaerobically through aortic cannulation. Kidneys were dissected into cortex and medulla on a cold plate (4° C.) then immediately frozen in liquid N₂ or frozen whole in liquid N₂ and subsequently stored at −80° C.

### Blood, Urine, and Fecal Analysis

Blood [Na⁺], [K⁺], and [Cl⁻] were measured with a Stat Profile pHOx Plus analyzer (Nova Biomedical; Waltham, Mass.) immediately after collection. Urine (24 hr) was collected under water equilibrated mineral oil and centrifuged at 1000×g to remove debris. Urine Na⁺ and K⁺ were measured by ion-sensitive electrodes (Nova 16 Analyzer; Nova Biomedical; Waltham, Mass.) or by analysis on a digital flame photometer (Cole Parmer, Model 2655-00) if less than the detectable limit for the ion-sensitive electrodes. Fecal samples were dried in an oven (200° C.) overnight, digested in 0.75M nitric acid overnight at 37° C. and homogenized. The samples were centrifuged and filtered to remove sediment. Fecal K⁺ content was determined by analysis of supernatants on a flame photometer using the linear standard curve method (Cole Parmer, Model 2655-00).

### Real Time PCR

Tissues were homogenized in TRIzol reagent (Invitrogen) to recover RNA according to the manufacturer's protocol. Subsequently, RNA was DNase-treated (DNA-Free, Ambion) and reverse transcribed into cDNA (SuperScript III, Invitrogen). For real time quantitative PCR, 20 ng cDNA was used to quantify mRNA expression with TaqMan Gene Expression (Applied Biosystems) primer/probe sets for several K⁺ channels and transporters with expression throughout the kidney. Cycle threshold (Ct) values were normalized to the endogenous control gene, β-actin (Actb Mm00607939_s1) and relative expression was calculated by the ΔΔCt method (Livak and Schmittgen, 2001). Fold change in mRNA expression was calculated relative to WT expression levels (set to 1).

### Statistical Analyses

SigmaPlot 12 was used to create graphs and perform statistical analyses. All data are presented as mean±standard error of the mean (SEM). One and two-way ANOVAs with or without repeated measures were performed to determine the significance of genotype, diet, and/or day on each dependent variable. If significance was found, then an appropriate post-hoc test was performed. Unpaired student's t-test was used to compare blood chemistries and mRNA expression data between genotypes. P values less than 0.05 were considered significant. For ANOVAs, the P values for the main effects and their interaction are displayed on the graph.

### Results

### K⁺ Homeostasis in WT and HKα Null Mice

To assess the effect of H⁺,K⁺-ATPase knockout on K⁺ homeostasis, blood [K⁺] was measured in WT and HKα_{1,2}^{-/-} mice fed either a control or K⁺ deplete diet for eight days. Blood [K⁺] was similar in WT and HKα_{1,2}^{-/-} mice on the control diet (Table 6.1). Although dietary K⁺ depletion led to hypokalemia in both WT and double knockout mice, the decrease in blood [K⁺] was an additional 1 mM in the knockouts. A separate study examined the effect of dietary K⁺ depletion on blood [K⁺] in HKα₁^{-/-} mice (Table 6.2). In contrast to the results from HKα_{1,2}^{-/-} mice, the drop in blood [K⁺] in HKα₁^{-/-} mice was not significantly different from that of WT mice.

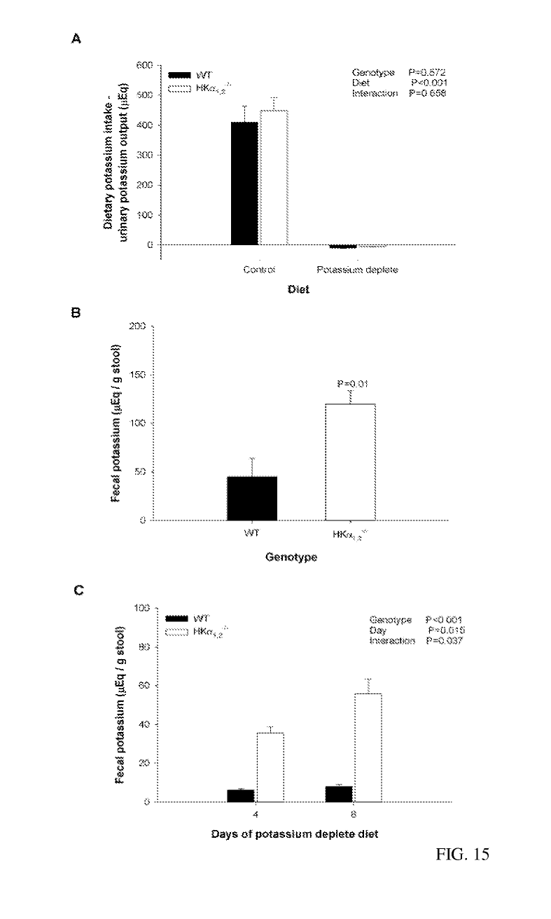

The next experiments sought to determine if the reduced blood [K⁺] observed in HKα_{1,2}^{-/-} mice resulted from urinary or fecal K⁺ loss. In order to correct for slight differences in food intake, all urinary excretion values are shown as the amount of the electrolyte (K⁺ or Na) consumed minus the amount excreted in the urine. The value corresponds to the amount of the electrolyte that the kidneys reabsorbed. On a control diet, urinary K⁺ reabsorption was identical in WT and HKα_{1,2}^{-/-} mice (FIG. 15A). With dietary K⁺ depletion, urinary K⁺ content was similarly reduced in WT and HKα_{1,2}^{-/-} mice. In contrast, fecal K⁺ wasting was quite apparent in HKα_{1,2}^{-/-} mice. On the control diet, HKα_{1,2}^{-/-} mice excreted nearly four-times more fecal K⁺ than WT mice (FIG. 15B). Although mice of both genotypes decreased fecal K⁺ output with dietary K⁺ depletion, fecal K⁺ excretion was still as much as seven-times greater in the HKα_{1,2}^{-/-} mice than WT mice on day 8 of dietary K⁺ depletion (FIG. 15C).

### Body Weight, Na⁺, and Fluid Homeostasis in WT and HKα Null Mice

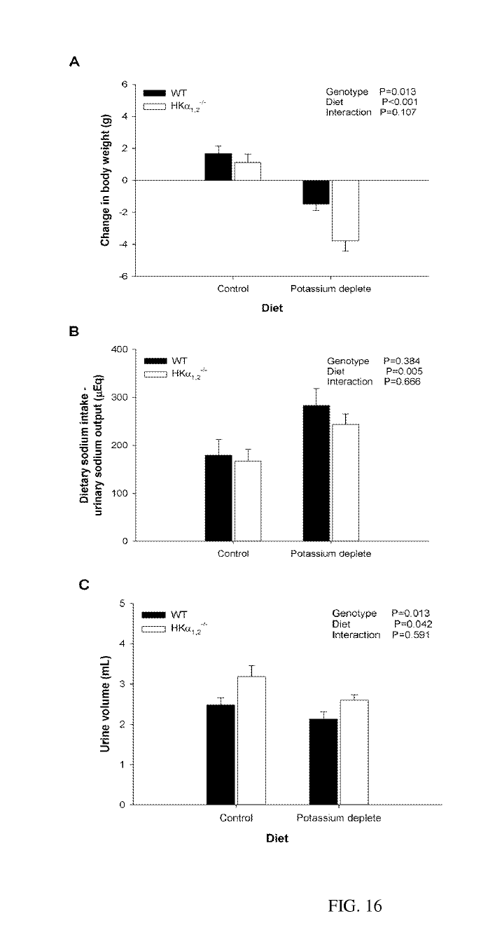

Previous studies have demonstrated a significant drop in body weight of the HKα₂^{-/-} mice subjected to dietary K⁺ depletion (Meneton et al., 1998). If HKα, partially compensated for loss of HKα₂, then mice null for both HKα, and HKα₂ might display exacerbated body weight loss with dietary K⁺ depletion. Therefore, changes in body weight in WT and HKα_{1,2} mice fed either a control or K⁺ deplete diet were followed for eight days. Body weight significantly decreased in HKα_{1,2}^{-/-} mice compared to WT mice from day 0 to day 8 (FIG. 16A). Although an interactions was not detected, dietary K⁺ depletion caused an ˜5% loss in body weight in WT mice and this effect appeared two-fold greater in HKα_{1,2}^{-/-} mice. The differences in body weight loss are comparable to previous observations in HKα₂^{-/-} mice (Meneton et al., 1998) and are consistent with loss of either body fluid or muscle mass.

The next experiment sought to determine whether the loss of body weight in K⁺-depleted HKα_{1,2}^{-/-} mice corresponded to greater urinary Na⁺ and fluid loss in the knockout mice compared to WT. Urinary Na⁺ content was measured in WT and HKα_{1,2}^{-/-} mice on the eighth day of a control or K⁺ depleted diet. Dietary K⁺ depletion stimulated urinary Na⁺ rebsorption to a similar extent in both WT and HKα_{1,2}^{-/-} mice (FIG. 16B), indicating that the body weight loss of the double knockouts does not result from urinary Na⁺ loss. However, urine volume was greater in HKα_{1,2}^{-/-} mice compared to WT mice regardless of whether the mice were fed a control or K⁺ depleted diet (FIG. 16C).

### Early Effects of Dietary K⁺ Depletion on Urine Na⁺ and K⁺ in HKα Null Mice

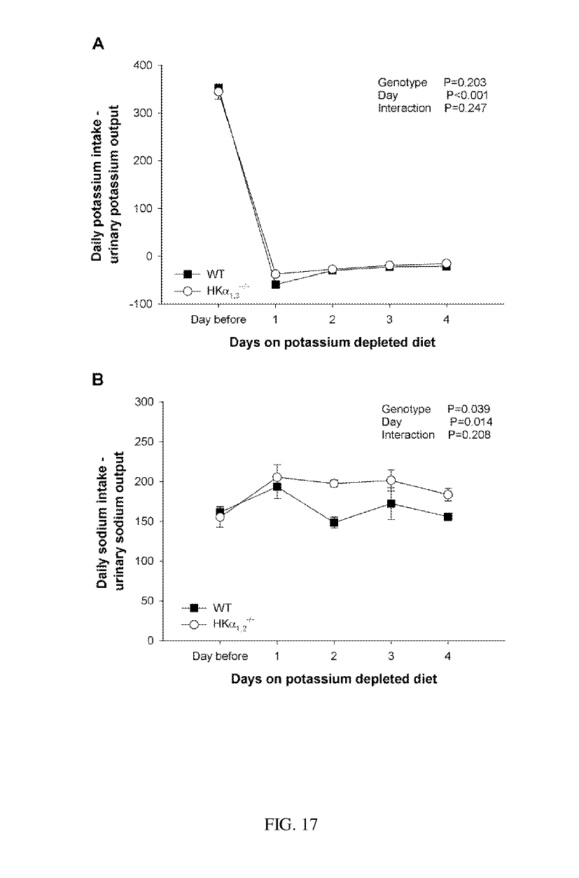

The studies described thus far primarily examined differences in K⁺ and Na⁺ balance on day 8 of a dietary K⁺ depleted diet. Therefore, the next experiments sought to determine if WT and HKα_{1,2}^{-/-} mice displayed significantly different urinary K⁺ or Na⁺ handling during the first four days of dietary K⁺ depletion. For these experiments, urine K⁺ and Na⁺ retention (FIGS. 17A and 17B, respectively) were compared in pair fed WT and HKα_{1,2}^{-/-} mice given a control diet for 4 days then switched to a K⁺ depleted diet for 4 days. Urinary K⁺ excretion was not different between WT and HKα_{1,2}^{-/-} mice over the first 4 days of dietary K⁺ depletion. However, the HKα_{1,2}^{-/-} mice exhibited slightly greater urinary Na retention over the entire four days of dietary K⁺ depletion.

### Discussion

In order to define the role of the H⁺,K⁺-ATPases in K⁺ homeostasis, the effect of dietary K⁺ depletion was investigated in WT, HKα, and HKα_{1,2}^{-/-} mice. It was expected that mice lacking both H,K-ATPase α subunits would be unable to efficiently reabsorb K⁺ from the urine particularly in the context of dietary K depletion thus leading to a hypokalemia. Although HKα_{1,2}^{-/-} mice did develop more severe hypokalemia than WT or HKα₁^{-/-} mice when fed a K⁺ depleted diet, urinary K⁺ excretion was not different between WT or HKα_{1,2}^{-/-} mice. However, the double knockouts did exhibit disproportionate fecal K⁺ excretion, indicating that the colonic HKα₂-containing H⁺,K⁺-ATPases are required for maximal K⁺ conservation under control and K⁺ depleted dietary conditions. Interestingly, HKα_{1,2}^{-/-} mice displayed greater urinary Na⁺ retention than WT mice during the first few days of dietary K⁺ depletion. This effect is in contrast to the inability of the mineralocorticoid, desoxycorticosterone pivalate, to stimulate urinary Na retention in HKα_{1,2}^{-/-} mice (Greenlee et al., 2011).

Considerable evidence has suggested that H⁺,K⁺-ATPases and, more specifically, HKα₂-containing H⁺,K⁺-ATPases mediate K⁺ reabsorption in the collecting duct and the colon (Greenlee et al., 2010; Gumz et al., 2010). Thus, the development of more severe hypokalemia in HKα_{1,2}^{-/-} mice than WT and HKα₁^{-/-} mice fed a K⁺ depleted diet was anticipated. The excessive fecal K⁺ wasting of HKα_{1,2}^{-/-} mice indicates that colonic HKα₂-containing H⁺,K⁺-ATPases facilitate maximal K⁺ conservation under K⁺ replete and deplete conditions. However, the observation that urinary K⁺ conservation mechanisms are intact in HKα_{1,2}^{-/-} mice as was observed in HKα₂^{-/-} mice (Meneton et al., 1998) suggests that other mechanisms in the kidney ably compensate for the lack of H⁺,K⁺-ATPase-mediated K⁺ reabsorption or that the renal H⁺,K⁺-ATPases do not participate in net K⁺ reabsorption in the context of dietary K⁺ depletion. Overall, our results reject the hypothesis that compensatory stimulation of HKα₁-containing H⁺,K⁺-ATPases allows for normal urinary K⁺ handling in HKα₂^{-/-} mice during dietary K⁺ depletion.

In other conditions, it appears that renal HKα₂-containing H⁺,K⁺-ATPases are involved in net K⁺ reabsorption. In a recent study, investigators observed that plasma progesterone levels increased during dietary K⁺ depletion in mice and that progesterone alone stimulated renal HKa2 mRNA expression and activity of HKα₂-containing H⁺,K⁺-ATPases in a cortical collecting duct cell line (Elabida et al., 2011). Most importantly and in contrast to WT mice, HKα₂^{-/-} mice did not exhibit a reduction in urinary K⁺/creatinine ratio with progesterone, indicating that renal HKα₂-containing H⁺,K⁺-ATPases are involved in net K⁺ reabsorption during activation by progesterone.

Previous studies show that mineralocorticoids did not cause urinary Na⁺ retention in HKα_{1,2}^{-/-} mice. Therefore, for this study, it was hypothesized that the urinary Na⁺ retention of dietary K⁺ depletion may also be dependent on the renal H⁺,K⁺-ATPases. However, dietary K⁺ depletion-induced Na⁺ retention was intact in HKα_{1,2}^{-/-} mice. The differences in the urinary Na⁺ retention of HKα_{1,2}^{-/-} mice during mineralocorticoid excess and dietary K⁺ depletion are possibly related to differences in mineralocorticoid status and the mechanisms responsible for Na⁺ reabsorption under these two conditions. Mineralocorticoid excess increases urinary Na⁺ reabsorption through mineralocorticoid stimulation of the epithelial sodium channel (ENaC) in principal cells of the collecting duct (Eaton et al., 2001). In contrast, diets low in K⁺ cause a drop in circulating mineralocorticoids (Hulter et al., 1980) and decrease ENaC subunit plasma membrane protein abundance in the cortical collecting duct (Frindt and Palmer, 2010). Thus, the lack of urinary Na retention in HKα_{1,2}^{-/-} mice during mineralocorticoid excess probably results from less activation of ENaC-mediated Na⁺ reabsorption. During dietary potassium depletion, this association between H⁺,K⁺-ATPases and ENaC is apparently not needed for maximal Na⁺ retention whereas other mechanisms like activation of the sodium chloride cotransporter are more likely involved (Frindt and Palmer, 2010).

In conclusion, the data presented herein re-illustrate that HKα₂-containing H⁺,K⁺-ATPases are required for maximal K⁺ conservation by the colonic epithelia. The lack of a significant renal phenotype in the HKα_{1,2}^{-/-} mice suggests that other, yet unknown, mechanisms are present in the kidney to compensate for the lack of K⁺ reabsorption by the H⁺,K⁺-ATPases.

### Tables for Example 6

TABLE 6.1
Blood chemistries of WT and HKα_{1,2}^{-/-} mice

| | Control diet (0.6% K⁺) | | K⁺ deplete diet (~0% K⁺) | |
|---|---|---|---|---|
| | | HKα_{1,2}^{-/-} | | HKα_{1,2}^{-/-} |
| | WT (n = 4) | (n = 5) | WT (n = 5) | (n = 4-6) |
| [Na⁺], mM | 148 ± 1.80 | 146 ± 1.60 | 150 ± 0.70 | 151 ± 1.80 |
| [K⁺], mM | 4.70 ± 0.13 | 4.46 ± 0.24 | 3.78 ± 0.21† | 2.81 ± 0.12*† |
| [Cl⁻], mM | 120 ± 1.10 | 119 ± 1.70 | 119 ± 2.60 | 120 ± 4.80 |

Two-way ANOVA with post-hoc Student-Newman-Keuls test - * denotes P < 0.05 versus WT and † P < 0.05 versus control diet.

TABLE 6.2
Blood chemistries of WT and HKα₁^{-/-} mice on a K⁺ deplete diet

| | WT (n = 4) | HKα₁^{-/-} (n = 4) |
|---|---|---|
| [Na⁺], mM | 147 ± 0.48 | 148 ± 0.47 |
| [K⁺], mM | 3.88 ± 0.10 | 3.75 ± 0.18 |
| [Cl⁻], mM | 114 ± 0.96 | 109 ± 0.89* |

Student's t-test - * denotes P < 0.05 versus WT.

### REFERENCES RELATED TO EXAMPLE 6

Adrogue, H. J., and Madias, N. E. (2007). Sodium and potassium in the pathogenesis of hypertension. N Engl J Med 356, 1966-1978.
Ahn, K. Y., Park, K. Y., Kim, K. K., and Kone, B. C. (1996a). Chronic hypokalemia enhances expression of the H(+)-K(+)-ATPase alpha 2-subunit gene in renal medulla. Am J Physiol 271, F314-321.
Ahn, K. Y., Turner, P. B., Madsen, K. M., and Kone, B. C. (1996b). Effects of chronic hypokalemia on renal expression of the “gastric” H(+)-K(+)-ATPase alpha-subunit gene. Am J Physiol 270, F557-566.
Codina, J., Delmas-Mata, J. T., and Dubose, T. D., Jr. (1998). Expression of HKalpha2 protein is increased selectively in renal medulla by chronic hypokalemia. Am J Physiol 275, F433-440.
Codina, J., Pressley, T. A., and Dubose, T. D., Jr. (1997). Effect of chronic hypokalemia on H(+)-K(+)-ATPase expression in rat colon. Am J Physiol 272, F22-30.
Coruzzi, P., Brambilla, L., Brambilla, V., Gualerzi, M., Rossi, M., Parati, G., Di Rienzo, M., Tadonio, J., and Novarini, A. (2001). Potassium depletion and salt sensitivity in essential hypertension. J Clin Endocrinol Metab 86, 2857-2862.
Coruzzi, P., Gualerzi, M., Parati, G., Brambilla, L., Brambilla, V., Di Rienzo, M., and Novarini, A. (2003). Potassium supplementation improves the natriuretic response to central volume expansion in primary aldosteronism. Metabolism 52, 1597-1600.
Doucet, A., and Marsy, S. (1987). Characterization of K-ATPase activity in distal nephron: stimulation by potassium depletion. Am J Physiol 253, F418-423.
Eaton, D. C., Malik, B., Saxena, N. C., Al-Khalili, O. K., and Yue, G. (2001). Mechanisms of aldosterone's action on epithelial Na+ transport. J Membr Biol 184, 313-319.
Elabida, B., Edwards, A., Salhi, A., Azroyan, A., Fodstad, H., Meneton, P., Doucet, A., Bloch-Faure, M., and Crambert, G. (2011). Chronic potassium depletion increases adrenal progesterone production that is necessary for efficient renal retention of potassium. Kidney Int 80, 256-262.
Frassetto, L. A., Schloetter, M., Mietus-Synder, M., Morris, R. C., Jr., and Sebastian, A. (2009). Metabolic and physiologic improvements from consuming a paleolithic, hunter-gatherer type diet. Eur J Clin Nutr 63, 947-955.
Frindt, G., and Palmer, L. G. (2010). Effects of dietary K on cell-surface expression of renal ion channels and transporters. Am J Physiol Renal Physiol 299, F890-897.
Greenlee, M. M., Lynch, I. J., Gumz, M. L., Cain, B. D., and Wingo, C. S. (2010). The renal H,K-ATPases. Curr Opin Nephrol Hypertens.
Greenlee, M. M., Lynch, I. J., Gumz, M. L., Cain, B. D., and Wingo, C. S. (2011).
Mineralocorticoids stimulate the activity and expression of renal H+,K+-ATPases. J Am Soc Nephrol.
Gumz, M. L., Lynch, I. J., Greenlee, M. M., Cain, B. D., and Wingo, C. S. (2010). The renal H+-K+-ATPases: physiology, regulation, and structure. Am J Physiol Renal Physiol 298, F12-21.
Hulter, H. N., Sebastian, A., Sigala, J. F., Licht, J. H., Glynn, R. D., Schambelan, M., and Biglieri, E. G. (1980). Pathogenesis of renal hyperchloremic acidosis resulting from dietary potassium restriction in the dog: role of aldosterone. Am J Physiol 238, F79-91
Kraut, J. A., Hiura, J., Besancon, M., Smolka, A., Sachs, G., and Scott, D. (1997). Effect of hypokalemia on the abundance of HK alpha 1 and HK alpha 2 protein in the rat kidney. Am J Physiol 272, F744-750.
Krishna, G. G., Chusid, P., and Hoeldtke, R. D. (1987). Mild potassium depletion provokes renal sodium retention. J Lab Clin Med 109, 724-730.
Krishna, G. G., and Kapoor, S. C. (1991). Potassium depletion exacerbates essential hypertension. Ann Intern Med 115, 77-83.
Krishna, G. G., and Kapoor, S. C. (1993). Potassium supplementation ameliorates mineralocorticoid-induced sodium retention. Kidney Int 43, 1097-1103.
Krishna, G. G., Miller, E., and Kapoor, S. (1989). Increased blood pressure during potassium depletion in normotensive men. N Engl J Med 320, 1177-1182.
Livak, K. J., and Schmittgen, T. D. (2001). Analysis of relative gene expression data using real-time quantitative PCR and the 2(−Delta Delta C(T)) Method. Methods 25, 402-408.
Lynch, I. J., Greenlee, M. M., Gumz, M. L., Rudin, A., Xia, S. L., and Wingo, C. S. (2010). Heterogeneity of H-K-ATPase-mediated acid secretion along the mouse collecting duct. Am J Physiol Renal Physiol 298, F408-415.
Lynch, I. J., Rudin, A., Xia, S. L., Stow, L. R., Shull, G. E., Weiner, I. D., Cain, B. D., and Wingo, C. S. (2008). Impaired acid secretion in cortical collecting duct intercalated cells from H-K-ATPase-deficient mice: role of HKalpha isoforms. Am J Physiol Renal Physiol 294, F621-627.
Meneton, P., Schultheis, P. J., Greeb, J., Nieman, M. L., Liu, L. H., Clarke, L. L., Duffy, J. J., Doetschman, T., Lorenz, J. N., and Shull, G. E. (1998). Increased sensitivity to K+ deprivation in colonic H,K-ATPase-deficient mice. J Clin Invest 101, 536-542.
Ray, P. E., Suga, S., Liu, X. H., Huang, X., and Johnson, R. J. (2001). Chronic potassium depletion induces renal injury, salt sensitivity, and hypertension in young rats. Kidney Int 59, 1850-1858.
Sweiry, J. H., and Binder, H. J. (1990). Active potassium absorption in rat distal colon. J Physiol 423, 155-170.
Wingo, C. S. (1989). Active proton secretion and potassium absorption in the rabbit outer medullary collecting duct. Functional evidence for proton-potassium-activated adenosine triphosphatase. J Clin Invest 84, 361-365.

It should be borne in mind that all patents, patent applications, patent publications, technical publications, scientific publications, and other references referenced herein are hereby incorporated by reference in this application in order to more fully describe the state of the art to which the present invention pertains.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method of screening for therapeutic agents useful in the treatment of elevated blood pressure comprising the steps of i) contacting a test compound with a HK alpha polypeptide or its mRNA, ii) detecting binding of said test compound to, or modulation of activity of, said HK alpha polypeptide or its mRNA, wherein a test compound which binds to said HK alpha polypeptide or its mRNA is identified as a potential therapeutic agent for controlling blood pressure.

2. (canceled)

3. (canceled)

4. The method of claim 1, wherein the step of contacting is in or at the surface of a cell.

5. The method of claim 1, wherein the cell is in vitro.

6. The method of claim 1, wherein the step of contacting is in a cell-free system.

7. The method of claim 1, wherein the polypeptide or its mRNA is coupled to a detectable label.

8. The method of claim 1, wherein the compound is coupled to a detectable label.

9. The method of claim 1, wherein the test compound displaces a ligand which is first bound to the polypeptide.

10. The method of claim 1, wherein the polypeptide is attached to a solid support.

11. The method of claim 1, wherein the compound is attached to a solid support.

12. A method of screening for therapeutic agents useful in the treatment of elevated blood pressure in a mammal comprising the steps of i) contacting a test compound with a HK alpha polynucleotide, ii) detecting binding of said test compound to said HK alpha polynucleotide.

13. The method of claim 12 wherein the polynucleotide is RNA.

14. The method of claim 12 wherein the contacting step is in or at the surface of a cell.

15. The method of claim 12 wherein the contacting step is in a cell-free system.

16. The method of claim 12 wherein polynucleotide is coupled to a detectable label.

17. The method of claim 12 wherein the test compound is coupled to a detectable label.

18. (canceled)

19. (canceled)

20. A pharmaceutical composition for the treatment of elevated blood pressure comprising a therapeutic agent which regulates the activity of a HK alpha polypeptide, wherein said therapeutic agent is i) a small molecule, ii) an RNA molecule, iii) an antisense oligonucleotide, iv) a polypeptide, v) an antibody, and vi) a ribozyme targeted to disrupt expression of HK alpha or disrupt activity of HK alpha.

21. The composition of claim 20, wherein said RNA molecule is siRNA, microRNA, shRNA

22. (canceled)

23. (canceled)

24. A method of treating elevated blood pressure, said method comprising administering a therapeutically effective amount of a composition according to claim 20 to a subject in need thereof.

25. (canceled)

26. (canceled)

27. (canceled)

28. (canceled)

29. (canceled)

30. (canceled)


SEQUENCE LISTING (8 sequences)

SEQ ID NO: 1 - protein, 1045 aa, Human
MHQKTPEIYSVELSGTKDIVKTDKGDGKEKYRGLKNNCLELKKKNHKEEFQKELHLDDHKLSNRELEEKYGTDIIMGLSSTRAAELLARDGPNSLTPPKQTPEIVKFLKQMVGGFSILLWVGAFLCWIAYGIQYSSDKSASLNNVYLGCVLGLVVILTGIFAYYQEAKSTNIMSSFNKMIPQQALVIRDSEKKTIPSEQLVVGDIVEVKGGDQIPADIRVLSSQGCRVDNSSLTGESEPQPRSSEFTHENPLETKNICFYSTTCLEASTSPVGTVTGMVINTGDRTIIGHIASLASGVGNEKTPIAIEIEHFVHIVAGVAVSIGILFFIIAVSLKYQVLDSIIFLIGIIVANVPEGLLATVTVTLSLTAKRMAKKNCLVKNLEAVETLGSTSIICSDKTGTLTQNRMTVAHLWFDNQIFVADTSEDHSNQVFDQSSRTWASLSKIITLCNRAEFKPGQENVPIMKKAVIGDASETALLKFSEVILGDVMEIRKRNRKVAEIPFNSTNKFQLSIHEMDDPHGKRFLMVMKGAPERILEKCSTIMINGEEHPLDKSTAKTFHTAYMELGGLGERVLGFCHLYLPADEFPETYSFDIDAMNFPTSNLCFVGLLSMIDPPRSTVPDAVTKCRSAGIKVIMVTGDHPITAKAIAKSVGIISANSETVEDIAHRLNIAVEQVNKRDAKAAVVTGMELKDMSSEQLDEILANYQEIVFARTSPQQKLIIVEGCQRQDAVVAVTGDGVNDSPALKKADIGIAMGIAGSDAAKNAADMVLLDDNFASIVTGVEEGRLIFDNLKKTIAYSLTKNIAELCPFLIYIIVGLPLPIGTITILFIDLGTDIIPSIALAYEKAESDIMNRKPRHKNKDRLVNQPLAVYSYLHIGLMQALGAFLVYFTVYAQEGFLPRTLINLRVEWEKDYVNDLKDSYGQEWTRYQREYLEWTGYTAFFVGILVQQIADLIIRKTRRNSIFQQGLFRNKVIWVGITSQIIIGLILSYGLGSVTALSFTMLRAQYWFVAVPHAILIWVYDEVRKLFIRLYPGSWWDKNMYY

SEQ ID NO: 2 - protein, 1039 aa, Human
MHQKTPEIYSVELSGTKDIVKTDKGDGKEKYRGLKNNCLELKKKNHKEEFQKELHLDDHKLSNRELEEKYGTDIIMGLSSTRAAELLARDGPNSLTPPKQTPEIVKFLKQMVGGFSILLWVGAFLCWIAYGIQYSSDKSASLNNVYLGCVLGLVVILTGIFAYYQEAKSTNIMSSFNKMIPQQALVIRDSEKKTIPSEQLVVGDIVEVKGGDQIPADIRVLSSQGCRVDNSSLTGESEPQPRSSEFTHENPLETKNICFYSTTCLEGTVTGMVINTGDRTIIGHIASLASGVGNEKTPIAIEIEHFVHIVAGVAVSIGILFFIIAVSLKYQVLDSIIFLIGIIVANVPEGLLATVTVTLSLTAKRMAKKNCLVKNLEAVETLGSTSIICSDKTGTLTQNRMTVAHLWFDNQIFVADTSEDHSNQVFDQSSRTWASLSKIITLCNRAEFKPGQENVPIMKKAVIGDASETALLKFSEVILGDVMEIRKRNRKVAEIPFNSTNKFQLSIHEMDDPHGKRFLMVMKGAPERILEKCSTIMINGEEHPLDKSTAKTFHTAYMELGGLGERVLGFCHLYLPADEFPETYSFDIDAMNFPTSNLCFVGLLSMIDPPRSTVPDAVTKCRSAGIKVIMVTGDHPITAKAIAKSVGIISANSETVEDIAHRLNIAVEQVNKRDAKAAVVTGMELKDMSSEQLDEILANYQEIVFARTSPQQKLIIVEGCQRQDAVVAVTGDGVNDSPALKKADIGIAMGIAGSDAAKNAADMVLLDDNFASIVTGVEEGRLIFDNLKKTIAYSLTKNIAELCPFLIYIIVGLPLPIGTITILFIDLGTDIIPSIALAYEKAESDIMNRKPRHKNKDRLVNQPLAVYSYLHIGLMQALGAFLVYFTVYAQEGFLPRTLINLRVEWEKDYVNDLKDSYGQEWTRYQREYLEWTGYTAFFVGILVQQIADLIIRKTRRNSIFQQGLFRNKVIWVGITSQIIIGLILSYGLGSVTALSFTMLRAQYWFVAVPHAILIWVYDEVRKLFIRLYPGSWWDKNMYY

SEQ ID NO: 3 - DNA, 3750 bp, Human
aatcctggactctcccgacccctagctgtcggtccccctccctgcgcgcgcgccggcgggtttcctaccctccgaggcgtccgctggcctgcgccctggcggggacgtgggcggggcgggcggcatttaaggctggtgccacctccccggtgcagcggctggcgatcggccgcggaggtgcgtgcagggcccgcgccgccgccggtatctccaccgccaacacctcagccactgccactgccacagccacacgaggccccccaccgtgcgctccgccgctgcggtcccggatccgcgctccacgcccgcagcccgcggcgccaccagcccagcatgcaccagaaaaccccagaaatttactccgtggagctcagcggaactaaggacatcgtgaaaacagacaagggggatggcaaggagaagtataggggtctgaagaacaactgcctggaactcaaaaagaaaaatcacaaagaggagtttcagaaagaactccatctggatgaccacaaactcagcaatagggaattggaagagaaatatggcacagacatcattatgggtctctccagcaccagagctgccgagctcctggcccgggatgggcccaactccctcacccctcccaagcagacgcctgagatcgtcaagttcctcaagcagatggtgggggggttctctatcctcctgtgggtgggcgcctttctctgttggattgcatatgggattcagtactccagcgacaagtctgcatccctgaacaacgtgtacttgggctgtgtgcttggtctggtggtcattttaacggggatctttgcttattaccaagaggcaaaaagcaccaacatcatgtccagcttcaataagatgatccctcagcaagctctcgtcatccgagattccgagaagaagaccatcccttcagagcagctggtggtgggggacattgtggaggtcaaaggaggagaccagatccctgcagacatcagggtgctgtcttctcaggggtgtcgggtggataactcatctctcacgggggagtctgagccccagccccgctcctctgagtttacccatgaaaaccccctggaaacaaagaacatctgcttctattccacaacgtgtctggaagcatctacttcccctgtaggcactgtcaccggcatggttatcaacacgggtgaccgcaccatcattggccatattgcctcattggcctcaggagttggaaatgagaagacgcccattgccattgagatcgagcactttgttcacattgtggcaggagtggctgtctccatcggcatccttttcttcatcatcgctgtgtccctgaagtatcaagtcctggactccatcatcttcctcattggcatcattgtggccaatgtgcccgagggcctcctggccactgtcactgtgaccctgtcgctgacagcaaaacggatggccaagaagaactgcctggtgaagaacctggaggctgtggagaccctcggctccacctccatcatctgctcggacaagactgggacactgacccagaacaggatgacagtggcccatctgtggttcgacaatcagatctttgtggctgacaccagtgaggaccattcaaaccaagtctttgaccaaagctctaggacttgggcctccttatccaagataataacattgtgtaaccgagcagagttcaagccaggacaggaaaatgtccccatcatgaagaaagctgtgattggagatgcctcagaaactgctcttttaaaattctcagaggtcattttgggtgatgtgatggaaattagaaaaagaaaccgcaaagtagctgaaatcccttttaactctactaataaatttcagctctccatccacgagatggatgacccccacggcaagcgcttcctcatggtgatgaagggggcccctgagcgcatcctagagaaatgcagcaccatcatgatcaacggcgaggagcacccactggacaagagcactgccaagaccttccacacagcctacatggagctgggcgggttgggcgagcgtgtgctgggtttctgtcatctctacctgccagcagacgagtttccagaaacctactcatttgacatagacgctatgaactttccgacctccaacctctgttttgtgggactcttgtcaatgatcgatccccctcggtccaccgtgccagatgcagtcaccaaatgccggagtgcagggatcaaggttattatggttactggtgatcatcccatcacagccaaagctattgccaagagtgtggggatcatttcagccaacagtgaaacagtggaagacattgcacatcgcctcaacattgctgtggagcaagttaacaaacgggatgccaaggccgctgtggtgactggcatggagctgaaggacatgagctcagaacagctggatgagatcttagccaactaccaggagattgtctttgcccggacatccccccagcagaagctgatcattgtggagggctgtcagaggcaggatgctgttgttgctgtgaccggggatggagttaatgactctccggctctaaagaaggcagacattgggattgccatggggatagcaggttctgatgcagccaaaaatgcagccgacatggtcttgctggacgacaacttcgcatccatcgtcacaggggtggaggaaggtcgcctgatctttgacaacctcaagaagactattgcttattccctgaccaagaacattgccgagctgtgcccctttctgatctacatcattgtcgggctccccctgcccattggcaccatcaccattctgttcattgacttggggacagacattatcccctccattgccttggcgtacgagaaagctgaaagtgacatcatgaacaggaagcctcgccacaagaataaggacaggctggtgaaccagccgctcgctgtgtactcatacctgcacattggcctcatgcaagccctgggagctttccttgtgtatttcaccgtctatgcacaagagggctttctgccccgcactctcattaacctgcgggtagaatgggagaaggactacgtgaatgacttgaaagacagctatgggcaggaatggacaaggtaccagagggaatacctagaatggacgggctacacggctttctttgttggcatcctagtccagcaaatagcagatctgatcatcaggaaaacccggaggaattccatcttccagcagggtctcttcagaaataaagtcatctgggtggggatcacctcacagatcatcattggtctgatcctctcctatggcctcggaagtgtcacagccttgagtttcaccatgcttagggctcagtactggtttgtggctgtgccgcacgccatcctgatctgggtgtatgatgaggtgcggaagctcttcatcaggctctaccctggaagctggtgggataagaacatgtattattaagaccacctcccttcctatgtctctcagcagcacgttggggcacacttgttcatcttctgaccgtttgctgggctattcccctgcagtgcagacatcgtcaaaattcagacaagaggaaattttcatgcagaaagctgtatgcaggatgctcactgatgttttgcactttaaaactgaaattcaactctttatataggattttcttttctatctccatctcctcattaaaaaatacgtacatttcgaggtaatggtatagggaaaaaaaaaaaaaaaaaa

SEQ ID NO: 4 - DNA, 3732 bp, Human
aatcctggactctcccgacccctagctgtcggtccccctccctgcgcgcgcgccggcgggtttcctaccctccgaggcgtccgctggcctgcgccctggcggggacgtgggcggggcgggcggcatttaaggctggtgccacctccccggtgcagcggctggcgatcggccgcggaggtgcgtgcagggcccgcgccgccgccggtatctccaccgccaacacctcagccactgccactgccacagccacacgaggccccccaccgtgcgctccgccgctgcggtcccggatccgcgctccacgcccgcagcccgcggcgccaccagcccagcatgcaccagaaaaccccagaaatttactccgtggagctcagcggaactaaggacatcgtgaaaacagacaagggggatggcaaggagaagtataggggtctgaagaacaactgcctggaactcaaaaagaaaaatcacaaagaggagtttcagaaagaactccatctggatgaccacaaactcagcaatagggaattggaagagaaatatggcacagacatcattatgggtctctccagcaccagagctgccgagctcctggcccgggatgggcccaactccctcacccctcccaagcagacgcctgagatcgtcaagttcctcaagcagatggtgggggggttctctatcctcctgtgggtgggcgcctttctctgttggattgcatatgggattcagtactccagcgacaagtctgcatccctgaacaacgtgtacttgggctgtgtgcttggtctggtggtcattttaacggggatctttgcttattaccaagaggcaaaaagcaccaacatcatgtccagcttcaataagatgatccctcagcaagctctcgtcatccgagattccgagaagaagaccatcccttcagagcagctggtggtgggggacattgtggaggtcaaaggaggagaccagatccctgcagacatcagggtgctgtcttctcaggggtgtcgggtggataactcatctctcacgggggagtctgagccccagccccgctcctctgagtttacccatgaaaaccccctggaaacaaagaacatctgcttctattccacaacgtgtctggaaggcactgtcaccggcatggttatcaacacgggtgaccgcaccatcattggccatattgcctcattggcctcaggagttggaaatgagaagacgcccattgccattgagatcgagcactttgttcacattgtggcaggagtggctgtctccatcggcatccttttcttcatcatcgctgtgtccctgaagtatcaagtcctggactccatcatcttcctcattggcatcattgtggccaatgtgcccgagggcctcctggccactgtcactgtgaccctgtcgctgacagcaaaacggatggccaagaagaactgcctggtgaagaacctggaggctgtggagaccctcggctccacctccatcatctgctcggacaagactgggacactgacccagaacaggatgacagtggcccatctgtggttcgacaatcagatctttgtggctgacaccagtgaggaccattcaaaccaagtctttgaccaaagctctaggacttgggcctccttatccaagataataacattgtgtaaccgagcagagttcaagccaggacaggaaaatgtccccatcatgaagaaagctgtgattggagatgcctcagaaactgctcttttaaaattctcagaggtcattttgggtgatgtgatggaaattagaaaaagaaaccgcaaagtagctgaaatcccttttaactctactaataaatttcagctctccatccacgagatggatgacccccacggcaagcgcttcctcatggtgatgaagggggcccctgagcgcatcctagagaaatgcagcaccatcatgatcaacggcgaggagcacccactggacaagagcactgccaagaccttccacacagcctacatggagctgggcgggttgggcgagcgtgtgctgggtttctgtcatctctacctgccagcagacgagtttccagaaacctactcatttgacatagacgctatgaactttccgacctccaacctctgttttgtgggactcttgtcaatgatcgatccccctcggtccaccgtgccagatgcagtcaccaaatgccggagtgcagggatcaaggttattatggttactggtgatcatcccatcacagccaaagctattgccaagagtgtggggatcatttcagccaacagtgaaacagtggaagacattgcacatcgcctcaacattgctgtggagcaagttaacaaacgggatgccaaggccgctgtggtgactggcatggagctgaaggacatgagctcagaacagctggatgagatcttagccaactaccaggagattgtctttgcccggacatccccccagcagaagctgatcattgtggagggctgtcagaggcaggatgctgttgttgctgtgaccggggatggagttaatgactctccggctctaaagaaggcagacattgggattgccatggggatagcaggttctgatgcagccaaaaatgcagccgacatggtcttgctggacgacaacttcgcatccatcgtcacaggggtggaggaaggtcgcctgatctttgacaacctcaagaagactattgcttattccctgaccaagaacattgccgagctgtgcccctttctgatctacatcattgtcgggctccccctgcccattggcaccatcaccattctgttcattgacttggggacagacattatcccctccattgccttggcgtacgagaaagctgaaagtgacatcatgaacaggaagcctcgccacaagaataaggacaggctggtgaaccagccgctcgctgtgtactcatacctgcacattggcctcatgcaagccctgggagctttccttgtgtatttcaccgtctatgcacaagagggctttctgccccgcactctcattaacctgcgggtagaatgggagaaggactacgtgaatgacttgaaagacagctatgggcaggaatggacaaggtaccagagggaatacctagaatggacgggctacacggctttctttgttggcatcctagtccagcaaatagcagatctgatcatcaggaaaacccggaggaattccatcttccagcagggtctcttcagaaataaagtcatctgggtggggatcacctcacagatcatcattggtctgatcctctcctatggcctcggaagtgtcacagccttgagtttcaccatgcttagggctcagtactggtttgtggctgtgccgcacgccatcctgatctgggtgtatgatgaggtgcggaagctcttcatcaggctctaccctggaagctggtgggataagaacatgtattattaagaccacctcccttcctatgtctctcagcagcacgttggggcacacttgttcatcttctgaccgtttgctgggctattcccctgcagtgcagacatcgtcaaaattcagacaagaggaaattttcatgcagaaagctgtatgcaggatgctcactgatgttttgcactttaaaactgaaattcaactctttatataggattttcttttctatctccatctcctcattaaaaaatacgtacatttcgaggtaatggtatagggaaaaaaaaaaaaaaaaaa

SEQ ID NO: 5 - protein, 1045 aa, Human; identical to SEQ ID NO: 1

SEQ ID NO: 6 - DNA, 3565 bp, Human
tggccgcggaggtgcgtgcagggcccgcgccgccgccggtatctccaccgccaacacctcagccactgccactgccacagccacacgaggccccccaccgtgcgctccgccgctgcggtcccggatccgcgctccacgcccgcagcccgcggcgccaccagcccagcatgcaccagaaaaccccagaaatttactccgtggagctcagcggaactaaggacatcgtgaaaacagacaagggggatggcaaggagaagtataggggtctgaagaacaactgcctggaactcaaaaagaaaaatcacaaagaggagtttcagaaagaactccatctggatgaccacaaactcagcaatagggaattggaagagaaatatggcacagacatcattatgggtctctccagcaccagagctgccgagctcctggcccgggatgggcccaactccctcacccctcccaagcagacgcctgagatcgtcaagttcctcaagcagatggtgggggggttctctatcctcctgtgggtgggcgcctttctctgttggattgcatatgggattcagtactccagcgacaagtctgcatccctgaacaacgtgtacttgggctgtgtgcttggtctggtggtcattttaacggggatctttgcttattaccaagaggcaaaaagcaccaacatcatgtccagcttcaataagatgatccctcagcaagctctcgtcatccgagattccgagaagaagaccatcccttcagagcagctggtggtgggggacattgtggaggtcaaaggaggagaccagatccctgcagacatcagggtgctgtcttctcaggggtgtcgggtggataactcatctctcacgggggagtctgagccccagccccgctcctctgagtttacccatgaaaaccccctggaaacaaagaacatctgcttctattccacaacgtgtctggaagcatctacttcccctgtaggcactgtcaccggcatggttatcaacacgggtgaccgcaccatcattggccatattgcctcattggcctcaggagttggaaatgagaagacgcccattgccattgagatcgagcactttgttcacattgtggcaggagtggctgtctccatcggcatccttttcttcatcatcgctgtgtccctgaagtatcaagtcctggactccatcatcttcctcattggcatcattgtggccaatgtgcccgagggcctcctggccactgtcactgtgaccctgtcgctgacagcaaaacggatggccaagaagaactgcctggtgaagaacctggaggctgtggagaccctcggctccacctccatcatctgctcggacaagactgggacactgacccagaacaggatgacagtggcccatctgtggttcgacaatcagatctttgtggctgacaccagtgaggaccattcaaaccaagtctttgaccaaagctctaggacttgggcctccttatccaagataataacattgtgtaaccgagcagagttcaagccaggacaggaaaatgtccccatcatgaagaaagctgtgattggagatgcctcagaaactgctcttttaaaattctcagaggtcattttgggtgatgtgatggaaattagaaaaagaaaccgcaaagtagctgaaatcccttttaactctactaataaatttcagctctccatccacgagatggatgacccccacggcaagcgcttcctcatggtgatgaagggggcccctgagcgcattctagagaaatgcagcaccatcatgatcaacggcgaggagcacccactggacaagagcactgccaagaccttccacacagcctacatggagctgggcgggttgggcgagcgtgtgctgggtttctgtcatctctacctgccagcagacgagtttccagaaacctactcatttgacatagacgctatgaactttccgacctccaacctctgttttgtgggactcttgtcaatgatcgatccccctcggtccaccgtgccagatgcagtcaccaaatgccggagtgcagggatcaaggttattatggttactggtgatcatcccatcacagccaaagctattgccaagagtgtggggatcatttcagccaacagtgaaacagtggaagacattgcacatcgcctcaacattgctgtggagcaagttaacaaacgggatgccaaggccgctgtggtgactggcatggagctgaaggacatgagctcagaacagctggatgagatcttagccaactaccaggagattgtctttgcccggacatccccccagcagaagctgatcattgtggagggctgtcagaggcaggatgctgttgttgctgtgaccggggatggagttaatgactctccggctctaaagaaggcagacattgggattgccatggggatagcaggttctgatgcagccaaaaatgcagccgacatggtcttgctggacgacaacttcgcatccatcgtcacaggggtggaggaaggtcgcctgatctttgacaacctcaagaagactattgcttattccctgaccaagaacattgccgagctgtgcccctttctgatctacatcattgtcgggctccccctgcccattggcaccatcaccattctgttcattgacttggggacagacattatcccctccattgccttggcgtacgagaaagctgaaagtgacatcatgaacaggaagcctcgccacaagaataaggacaggctggtgaaccagccgctcgctgtgtactcatacctgcacattggcctcatgcaagccctgggagctttccttgtgtatttcaccgtctatgcacaagagggctttctgccccgcactctcattaacctgcgggtagaatgggagaaggactacgtgaatgacttgaaagacagctatgggcaggaatggacaaggtaccagagggaatacctagaatggacgggctacacggctttctttgttggcatcctagtccagcaaatagcagatctgatcatcaggaaaacccggaggaattccatcttccagcagggtctcttcagaaataaagtcatctgggtggggatcacctcacagatcatcattggtctgatcctctcctatggcctcggaagtgtcacagccttgagtttcaccatgcttagggctcagtactggtttgtggctgtgccgcacgccatcctgatctgggtgtatgatgaggtgcggaagctcttcatcaggctctaccctggaagctggtgggataagaacatgtattattaagaccacctcccttcctatgtctctcagcagcacgttggggcacacttgttcatcttctgaccgtttgctgggctattcccctgcagtgcagacatcgtcaaaattcatacaagaggaaattttcatgcagaaagctgtatgcaggatgctcactgatgttttgcactttaaaactgaaattcaactctttatataggattttcttttctatctccatctcctcattaaaaaatacgtacatttcgaggtaaaaaaaaaaa

SEQ ID NO: 7 - protein, 1035 aa, Human
MGKAENYELYSVELGPGPGGDMAAKMSKKKKAGGGGGKRKEKLENMKKEMEINDHQLSVAELEQKYQTSATKGLSASLAAELLLRDGPNALRPPRGTPEYVKFARQLAGGLQCLMWVAAAICLIAFAIQASEGDLTTDDNLYLAIALIAVVVVTGCFGYYQEFKSTNIIASFKNLVPQQATVIRDGDKFQINADQLVVGDLVEMKGGDRVPADIRILAAQGCKVDNSSLTGESEPQTRSPECTHESPLETRNIAFFSTMCLEGTVQGLVVNTGDRTIIGRIASLASGVENEKTPIAIEIEHFVDIIAGLAILFGATFFIVAMCIGYTFLRAMVFFMAIVVAYVPEGLLATVTVCLSLTAKRLASKNCVVKNLEAVETLGSTSVICSDKTGTLTQNRMTVSHLWFDNHIHTADTTEDQSGQTFDQSSETWRALCRVLTLCNRAAFKSGQDAVPVPKRIVIGDASETALLKFSELTLGNAMGYRDRFPKVCEIPFNSTNKFQLSIHTLEDPRDPRHLLVMKGAPERVLERCSSILIKGQELPLDEQWREAFQTAYLSLGGLGERVLGFCQLYLNEKDYPPGYAFDVEAMNFPSSGLCFAGLVSMIDPPRATVPDAVLKCRTAGIRVIMVTGDHPITAKAIAASVGIISEGSETVEDIAARLRVPVDQVNRKDARACVINGMQLKDMDPSELVEALRTHPEMVFARTSPQQKLVIVESCQRLGAIVAVTGDGVNDSPALKKADIGVAMGIAGSDAAKNAADMILLDDNFASIVTGVEQGRLIFDNLKKSIAYTLTKNIPELTPYLIYITVSVPLPLGCITILFIELCTDIFPSVSLAYEKAESDIMHLRPRNPKRDRLVNEPLAAYSYFQIGAIQSFAGFTDYFTAMAQEGWFPLLCVGLRAQWEDHHLQDLQDSYGQEWTFGQRLYQQYTCYTVFFISIEVCQIADVLIRKTRRLSAFQQGFFRNKILVIAIVFQVCIGCFLCYCPGMPNIFNFMPIRFQWWLVPLPYGILIFVYDEIRKLGVRCCPGSWWDQELYY

SEQ ID NO: 8 - DNA, 3582 bp, Human
gttgggtgggagcacaggcaccgggcaccatggggaaggccgagaactatgagctctactcggtggagctgggtcctggccctggcggggacatggctgccaagatgagcaagaagaagaaggcgggtggcgggggtggcaagaggaaggagaagctggagaacatgaagaaggagatggagattaacgaccaccagctgtcagtggcggagctggaacagaaataccagaccagtgccaccaagggcctctctgcgagcctggctgctgagctgctgctgcgggatgggcccaacgcactgcggccaccacggggcaccccagagtacgtcaagttcgcgaggcagctggccgggggcctgcagtgcctcatgtgggttgccgccgccatctgcctcatcgcctttgccatccaggctagtgagggggacctcaccaccgacgacaatctgtacctggcaatcgctctcattgctgtggttgtcgtcaccggctgctttggctactaccaggaattcaagagcaccaacatcatcgccagctttaagaaccttgtgccacagcaagccactgtcatccgcgatggagacaaattccagatcaacgctgaccaactggtggtgggcgacctggtggagatgaaaggtggggacagagtgcccgccgacatccgcatcctggcggcccagggctgcaaggtggacaactcctcgctgacaggggagtctgagccacagacccgctcacccgagtgcacgcacgagagccctctggagacccgcaacatcgccttcttctccaccatgtgccttgagggcaccgtgcagggcctggtggtgaacacgggcgaccgcaccatcattgggcgcatcgcatcgctggcgtcgggggtggaaaacgagaagacacccatcgctatcgagatcgagcattttgtggacatcatcgcgggcctggccattctcttcggtgccacattttttattgtggccatgtgcattggctacaccttcctgcgggccatggtcttcttcatggccatcgtggtggcctatgtgcctgaggggctgctggccactgtcacagtctgcctgtccctgacagccaagcgcctggccagtaagaactgcgtggtcaagaacctggaggcggtggagacattgggctccacttcggtgatctgctcggacaagacagggactctcactcagaaccgcatgactgtgtcccatctgtggtttgacaaccacatccacacagctgacaccacggaagaccagtcagggcagacgtttgaccagtcctcggagacgtggcgggcgctgtgccgggtgctcaccctgtgcaaccgcgccgccttcaagtccggccaggatgcagtgcctgtgcccaagcgcatcgtgattggagacgcatcggagacggcgctgctcaagttctcggagctgacgctgggcaacgccatgggctaccgggaccgcttcccaaaagtctgcgagatacccttcaactccaccaacaagttccagctgtccatccatacgctggaggacccgcgggacccgcgacacttgctggtgatgaagggcgcccccgagcgcgtgctggagcgctgcagctccatccttatcaagggccaggagctgccgctggacgagcagtggcgcgaggccttccagaccgcctacctcagcctgggaggcctgggcgaacgcgtgctcggcttctgccagctctacctgaatgagaaggactacccgcctggctatgccttcgacgtagaggccatgaactttccatctagcggcctctgctttgcgggacttgtatccatgattgacccaccccgggccaccgtccctgatgctgtgctcaagtgtcgcaccgcaggcatccgggtgatcatggtaacgggtgaccaccccatcaccgccaaggccattgcagccagtgtgggcatcatctcggaaggcagcgagacagtggaggacatcgctgcccgcctccgtgtgcccgtagaccaggttaatcgcaaggatgcccgtgcctgtgtgatcaatggcatgcagctgaaggacatggacccatcggaactggtcgaggccctgcgcacccaccccgagatggtgtttgcgcgcaccagcccccagcagaagctggtgatcgtggagagctgccagcggctgggtgcgattgtggccgtcacgggggatggtgtgaatgactccccagctctgaagaaggcagacatcggagtagccatgggcatcgctggctcagatgctgccaaaaatgcagctgacatgatcctgctggatgacaactttgcctccattgtgacaggcgtggagcagggtcgactgatcttcgacaacctgaagaagtctattgcctacacattgaccaagaacatcccagagctgacaccctacctcatctacatcaccgtcagcgtgcccctgcccctcgggtgcatcaccatcctcttcatcgaactctgcactgacattttcccatctgtgtccctggcatatgaaaaggccgagagtgacatcatgcacctgcgtccacgcaacccaaagcgtgacagattggtcaacgagcccctggctgcctactcctacttccagattggtgccattcagtcctttgctggcttcactgactacttcacggcaatggcccaggagggctggttcccactgctgtgcgtggggctgcgggcgcagtgggaggaccaccacctacaagatctgcaggacagctacggccaggagtggacattcgggcagcgcctgtaccagcagtacacctgctacaccgtgttcttcatcagcattgaggtgtgccagatcgccgatgtcctcatccgcaagacgcgccgtctctctgccttccagcaaggcttcttcaggaataagatcctggtgatcgccatcgtgttccaggtctgcatcggctgcttcctgtgctactgccccggcatgcccaacatcttcaacttcatgcccattcggttccagtggtggctggtccccctgccctacggcatcctcatcttcgtctatgatgagatccggaagcttggagttcgctgttgcccagggagctggtgggaccaggaactctactattagagggacgactgccttcaagcatccctgcaactgccacagcaggtgggggcagggctcgtgggaccctctggacagccaccaagatatctgagcaaccaagagtcccagccccaccagtatctgcttctgtagcccacggcaccccaaacttggagggacctgcccactcccctcccccattcccaaggttcgcacctcctggagcagcagcgcctgggcagtcctctgggctggcctcgggaaagccgccacctgtggtggcggtggggctctgacagggagtacagctgaccgcttctggagggtgtttctgttcttaggactccagtccaggctggacggctgcctgagggcccttcgttaaagacacgcttgtgtcctgggcgatggtaataaaaccagctcatgctgactgtgctgtaaaaaaaaaaaaaaaaaaaaaaaa